SEQUENCE LISTING (6 sequences)

SEQ ID NO: 1 - DNA, 7650 bp, Homo sapiens
  CDS (1)...(7650)
atgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaa

SEQ ID NO: 2 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 3 - DNA, 1026 bp, Homo sapiens
  CDS (1)...(1026)
atgcaggagggagaattggctatttctcctataagccctgtggcagccatgcctcccctaggcacccacgtgcaagccagatgtgaagctcaaattaacctgctgggtgaaggggggatctgcaagctgccaggaagactccgcatccagcccgcactgtggagcagggaggacgtgctgcactggctgcgctgggcagagcaggagtactctctgccatgcaccgcggagcacgggttcgagatgaacggacgcgccctctgcatcctcaccaaggacgacttccggcaccgtgcgcccagctcaggtgacgtcctgtatgagctgctccagtacatcaagacccagcggcgagccctggtgtgtggacccttttttggagggatcttcaggctgaagacgcccacccagcactctccagtccccccggaagaggtgactggcccctctcagatggacacccgaaggggccacctgctgcagccaccagacccagggcttaccagcaacttcggccacctggatgaccctggcctggcaaggtggacccctggcaaggaggagtccctcaacttatgtcactgtgcagagctcggctgcaggacccagggggtctgttccttccccgcgatgccgcaggcccccattgacggcaggatcgctgactgccgcctgctgtgggattacgtgtatcagctgctccttgatacccgatatgagccctacatcaagtgggaagacaaggacgccaagatcttccgagttgtggatccaaatgggctcgccagactctggggaaatcacaagaaccgggtgaacatgacctacgagaagatgtctcgtgccctgcgccactattataagcttaatatcattaagaaggaaccggggcagaaactcctgttcagatttctaaagactccgggaaagatggtccaggacaagcacagccacctggagccgctggagagccaggagcaggacagaatagagttcaaggacaagaggccagaaatctctccgtga

SEQ ID NO: 4 - protein, 341 aa, Homo sapiens
MQEGELAISPISPVAAMPPLGTHVQARCEAQINLLGEGGICKLPGRLRIQPALWSREDVLHWLRWAEQEYSLPCTAEHGFEMNGRALCILTKDDFRHRAPSSGDVLYELLQYIKTQRRALVCGPFFGGIFRLKTPTQHSPVPPEEVTGPSQMDTRRGHLLQPPDPGLTSNFGHLDDPGLARWTPGKEESLNLCHCAELGCRTQGVCSFPAMPQAPIDGRIADCRLLWDYVYQLLLDTRYEPYIKWEDKDAKIFRVVDPNGLARLWGNHKNRVNMTYEKMSRALRHYYKLNIIKKEPGQKLLFRFLKTPGKMVQDKHSHLEPLESQEQDRIEFKDKRPEISP

SEQ ID NO: 5 - DNA, 357 bp, Homo sapiens
  CDS (1)...(357)
atgtccgggggcagcagctgcagccagaccccaagccgggccatccccgccactcgccgggtggtgctcggcgacggcgtgcagctcccgcccggggactacagcacgacccccggcggcacgctcttcagcaccaccccgggaggtaccaggatcatctatgaccggaaattcctgatggagtgtcggaactcacctgtgaccaaaacacccccaagggatctgcccaccattccgggggtcaccagcccttccagtgatgagccccccatggaagccagccagagccacctgcgcaatagcccagaagataagcgggcgggcggtgaagagtcacagtttgagatggacatttaa

SEQ ID NO: 6 - protein, 118 aa, Homo sapiens
MSGGSSCSQTPSRAIPATRRVVLGDGVQLPPGDYSTTPGGTLFSTTPGGTRIIYDRKFLMECRNSPVTKTPPRDLPTIPGVTSPSSDEPPMEASQSHLRNSPEDKRAGGEESQFEMDI

(19) United States
(12) Patent Application Publication
(10) Pub. No.: US 2014/0157443 A1
(43) Pub. Date: Jun. 5, 2014

(54) METHODS AND COMPOSITIONS FOR DETECTING AND MODULATING A NOVEL MTOR COMPLEX

(71) Applicants: Gerard C. Grosveld, Memphis, TN (US); Frank C. Harwood, Coldwater, MS (US); Ramon I. Klein-Geltink, Memphis, TN (US)

(72) Inventors: Gerard C. Grosveld, Memphis, TN (US); Frank C. Harwood, Coldwater, MS (US); Ramon I. Klein-Geltink, Memphis, TN (US)

(73) Assignee: ST. JUDE CHILDREN'S RESEARCH HOSPITAL, Memphis, TN (US)

(21) Appl. No.: 14/111,447

(22) PCT Filed: Apr. 12, 2012
(86) PCT No.: PCT/US2012/033239

§ 371 Date: Jan. 13, 2014

Related U.S. Application Data
(60) Provisional application No. 61/475,456, filed on Apr. 14, 2011.

(51) Int. Cl. C07K 16/40; C12Q 1/68; A01K 67/027; A61K 31/5377; A61K 31/4709; C12N 9/12; G01N 33/574

(52) U.S. Cl. CPC C07K 16/40; C12N 9/12; C12Q 1/6886; G01N 33/57496; A61K 31/5377; A61K 31/4709; A01K 67/0275
USPC 800/10; 435/188; 530/387.9; 530/387.3; 424/139.1; 546/177; 544/80; 435/6.12; 435/6.11; 435/7.4; 435/15; 514/232.5; 514/314

(57) ABSTRACT

Provided herein is a novel mTOR-comprising complex, mT-ORC3, which comprises mTOR and the Ets transcription factor TEL2. Specific mTORC3 binding agents and modulating agents are provided, along with kits and methods for the detection of mTORC3. Methods of modulating the activity of mTORC3 or modulating cell growth and/or survival are also provided. Further provided are methods for screening for mTORC3 binding agents and for mTORC3 modulating agents. Various methods of diagnosis and treatment are further provided.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 417922SEQLIST.TXT, created on Apr. 4, 2012, and having a size of 61.2 kilobytes and is filed concurrently with the specification. The sequence listing contained in this ASCII formatted document is part of the specification and is herein incorporated by reference in its entirety.

### FIELD OF THE INVENTION

The present invention relates to methods for regulating cell growth and survival, particularly through the modulation of the activity of an mTOR-comprising complex.

### BACKGROUND OF THE INVENTION

Cell growth and survival is regulated through a complex network of signaling pathways that are responsive to various cellular and environmental changes, including cell size, nutrient availability, oxygen, amino acid levels, and growth factors. The mammalian target of rapamycin (mTOR) has emerged as a growth regulatory signaling component that is a master effector of many cell signaling pathways in response to multiple cellular and environmental signals (Guertin and Sabatini (2007) Cancer Cell 12:9). mTOR is a PI3K-related kinase that regulates cell growth through the control of ribosome biogenesis, translation of mRNAs, metabolism, cytoskeleton organization and autophagy (Guertin and Sabatini (2005) Trends Mol Med 11:353).

To date, two mTOR protein complexes with non-overlapping kinase activity have been described: mTORC1, which contains mLST8, Raptor, DEPTOR and PRAS40; and mTORC2, which contains mLST8, SIN1, Rictor, DEPTOR, and Protor/PRR5 or PPR5L (Hara et al. (2002) Cell 110:177; Jacinto et al. (2004) Nat Cell Biol 6:1122; Kim et al. (2002) Cell 110:163; Loewith et al. (2002) Mol Cell 10:457; Pearce et al. (2007) Biochem J 405:513; Sarbassov et al. (2004) Curr Biol 14:1296; Thedieck et al. (2007) PLoS ONE 2:e1217; Woo et al. (2007) J Biol Chem 282:25604). These complexes target different substrates; mTORC1 phosphorylates the protein synthesis regulators p70S6K and 4E-BP1 (Brunn et al. (1997) Science 277:99; Burnett et al. (1998) Proc Natl Acad Sci USA 95:1432), while mTORC2 phosphorylates AGC kinases, including Akt at Ser-473, protein kinase Cα (PKCα) at Ser-657 (Zoncu et al. (2011) Nat Rev Mol Cell Biol 12:21-35) and the serum and glucocorticoid induced protein kinase-1 (SGK-1) at Ser-422 (Garcia-Martinez and Alessi (2008) Biochem J416:375). mTORC2 is also possibly involved in regulation of the actin cytoskeleton (Jacinto et al. (2004) Nat Cell Biol 6:1122; Sarbassov et al. (2004) Curr Biol 14:1296; Guertin et al. (2006) Dev Cell 11:859; Hresko and Mueckler (2005) J Biol Chem 280:40406).

Given the important role that mTOR plays in integrating various cellular and environmental signals to regulate cell growth, proliferation, and survival and the frequent dysregulation of mTOR in diseases associated with unregulated growth, such as cancer, there is a need for a better understanding of the complexes through which mTOR functions and for agents that can modulate the activity of mTOR.

### BRIEF SUMMARY OF THE INVENTION

A novel mTOR-comprising complex, the mTOR complex 3 (mTORC3), which also comprises the Ets transcription factor TEL2 is described. Various compositions and methods for detecting the mTORC3 and modulating its activity or modulating cell growth and/or survival are provided. Methods of diagnosis and treatment of cancers through the administration of specific mTORC3 antagonists or TEL2 antagonists are also provided. Further provided are methods for screening for mTORC3 binding agents and for agents that modulate the activity of mTORC3.

The following embodiments are encompassed by the present invention:

1. An isolated mTOR complex 3 (mTORC3), wherein said mTORC3 comprises:
a) a first polypeptide comprising an mTOR polypeptide or a biologically active variant or fragment thereof; and
b) a second polypeptide comprising a TEL2 polypeptide or a biologically active variant or fragment thereof.

2. The isolated mTORC3 of embodiment 1, wherein said first polypeptide comprises the mTOR polypeptide of SEQ ID NO: 2, a biologically active fragment thereof, or a biologically active variant thereof having at least 80% sequence identity to the mTOR polypeptide of SEQ ID NO: 2.

3. The isolated mTORC3 of embodiment 1 or 2, wherein said second polypeptide comprises the TEL2 polypeptide of SEQ ID NO: 4, a biologically active fragment thereof, or a biologically active variant thereof having at least 80% sequence identity to the TEL2 polypeptide of SEQ ID NO: 4.

4. The isolated mTORC3 of any one of embodiments 1-3, wherein said mTORC3 has a molecular weight greater than 1.5 MDa.

5. The isolated mTORC3 of any one of embodiments 1-4, wherein said mTORC3 further comprises 4E-BP1.

6. An antibody that specifically binds to an mTOR complex 3 (mTORC3), wherein said mTORC3 comprises:

a) a first polypeptide comprising an mTOR polypeptide or a biologically active variant or fragment thereof; and

b) a second polypeptide comprising a TEL2 polypeptide or a biologically active variant or fragment thereof.

7. The antibody of embodiment 6, wherein said first polypeptide comprises the mTOR polypeptide of SEQ ID NO: 2, a biologically active fragment thereof, or a biologically active variant thereof having at least 80% sequence identity to the mTOR polypeptide of SEQ ID NO: 2.

8. The antibody of embodiment 6 or 7, wherein said second polypeptide comprises the TEL2 polypeptide of SEQ ID NO: 4, a biologically active fragment thereof, or a biologically active variant thereof having at least 80% sequence identity to the TEL2 polypeptide of SEQ ID NO: 4.

9. The antibody of any one of embodiments 6-8, wherein said mTORC3 further comprises 4E-BP 1.

10. The antibody of any one of embodiments 6-9, wherein said antibody is a monoclonal antibody.

11. The antibody of any one of embodiments 6-9, wherein said antibody is bispecific, wherein a first antigen binding domain specifically interacts with said first polypeptide and said second antigen binding domain specifically interacts with said second polypeptide.

12. The antibody of any one of embodiments 6-11, wherein said antibody specifically inhibits the activity of an mTOR complex 3.

13. A mixture of a first and a second antibody comprising:

a) a first antibody having a first chemical moiety, wherein said first antibody specifically binds to a first polypeptide comprising an mTOR polypeptide or a biologically active variant or fragment thereof; and,

b) a second antibody having a second chemical moiety, wherein said second antibody specifically binds to a second polypeptide comprising a TEL2 polypeptide or a biologically active variant or fragment thereof;

wherein said first and said second chemical moiety allow for the detection of an mTOR complex 3 (mTORC3).

14. The mixture of said first and said second antibody of embodiment 13, wherein said first polypeptide comprises the mTOR polypeptide of SEQ ID NO: 2, a biologically active fragment thereof, or a biologically active variant thereof having at least 80% sequence identity to the mTOR polypeptide of SEQ ID NO: 2.

15. The mixture of said first and said second antibody of embodiment 13 or 14, wherein said second polypeptide comprises the TEL2 polypeptide of SEQ ID NO: 4, a biologically active fragment thereof, or a biologically active variant thereof having at least 80% sequence identity to the TEL2 polypeptide of SEQ ID NO: 4.

16. A compound that specifically inhibits the activity of an mTOR complex 3.

17. The compound of embodiment 16, wherein said compound comprises a small molecule.

18. A pharmaceutical composition comprising the antibody of any one of embodiments 6-12, the mixture of a first and a second antibody of any one of embodiments 13-15, or the compound of embodiment 16 or 17, and a pharmaceutically acceptable carrier.

19. A kit for determining the level of expression of a polynucleotide encoding an mTOR polypeptide and a polynucleotide encoding a TEL2 polypeptide in a sample comprising:

a) a first polynucleotide or pair of polynucleotides capable of specifically detecting or specifically amplifying a polynucleotide encoding an mTOR polypeptide or a biologically active variant or fragment thereof; and

b) a second polynucleotide or pair of polynucleotides capable of specifically detecting or specifically amplifying a polynucleotide encoding a TEL2 polypeptide or a biologically active variant or fragment thereof;

wherein the encoded polypeptides are capable of associating with one another in an mTOR complex 3 (mTORC3).

20. The kit of embodiment 19, wherein

a) the first polynucleotide or pair of polynucleotides is capable of specifically detecting or amplifying a polynucleotide encoding the amino acid sequence of SEQ ID NO:2 or a sequence having at least 80% sequence identity to SEQ ID NO:2; and,

b) the second polynucleotide or pair of polynucleotides is capable of specifically detecting or amplifying a polynucleotide encoding the amino acid sequence of SEQ ID NO:4 or a sequence having at least 80% sequence identity to SEQ ID NO:4.

21. The kit of embodiment 19 or 20, wherein:

a) said first pair of polynucleotides comprises a first and a second primer that share sufficient sequence homology or complementarity to said polynucleotide encoding an mTOR polypeptide or biologically active variant or fragment thereof to specifically amplify said polynucleotide encoding an mTOR polypeptide or biologically active variant or fragment thereof; and

b) said second pair of polynucleotides comprises a third and a forth primer that share sufficient sequence homology or complementarity to said polynucleotide encoding an TEL2 polypeptide or biologically active variant or fragment thereof to specifically amplify said polynucleotide encoding a TEL2 polypeptide or biologically active variant or fragment thereof.

22. The kit of embodiment 19 or 20, wherein said kit comprises:

a) a first polynucleotide that can specifically detect said polynucleotide encoding an mTOR polypeptide or biologically active variant or fragment thereof, wherein said first polynucleotide comprises at least one DNA molecule of a sufficient length of contiguous nucleotides identical or complementary to SEQ ID NO:1; and

b) a second polynucleotide that can specifically detect said polynucleotide encoding a TEL2 polypeptide or biologically active variant or fragment thereof, wherein said second polynucleotide comprises at least one DNA molecule of a sufficient length of contiguous nucleotides identical or complementary to SEQ ID NO:3.

23. The kit of embodiment 19 or 20, wherein said kit comprises

a) a first polynucleotide that hybridizes under stringent conditions to the sequence of SEQ ID NO:1; and

b) a second polynucleotide that hybridizes under stringent conditions to the sequence of SEQ ID NO:3.

24. A kit for detecting the presence of an mTOR complex 3 (mTORC3) in a sample comprising an antibody of any one of embodiments 6-12 or the mixture of a first and a second antibody of any one of embodiments 13-15.

25. A method for detecting the level of expression of a polynucleotide encoding an mTOR polypeptide and a polynucleotide encoding a TEL2 polypeptide in a sample comprising

a) contacting said sample with
i) a first and a second primer capable of specifically amplifying a first amplicon of a polynucleotide encoding an mTOR polypeptide or a biologically active variant or fragment thereof and,
ii) a third and a fourth primer capable of specifically amplifying a second amplicon of a polynucleotide encoding a TEL2 polypeptide or a biologically active variant or fragment thereof;

wherein the encoded polypeptides are capable of associating with one another in an mTOR complex 3 (mTORC3);

b) amplifying said first and said second amplicon; and

c) detecting said first and said second amplicon and thereby detecting the level of expression of a polynucleotide encoding an mTOR polypeptide and a polynucleotide encoding a TEL2 polypeptide in said sample.

26. The method of embodiment 25, wherein said first and said second primer comprise at least 8 consecutive polynucleotides of SEQ ID NO: 1 or the complement thereof, and said third and said fourth primer comprise at least 8 consecutive polynucleotides of SEQ ID NO:3 or the complement thereof.

27. A method for detecting the level of expression of a polynucleotide encoding an mTOR polypeptide and a polynucleotide encoding a TEL2 polypeptide in a sample, said method comprising:

a) contacting said sample with
i) a first polynucleotide capable of specifically detecting a polynucleotide encoding an mTOR polypeptide or a biologically active variant or fragment thereof; and,
ii) a second polynucleotide capable of specifically detecting a polynucleotide encoding a TEL2 polypeptide or a biologically active variant or fragment thereof;

wherein the encoded polypeptides are capable of associating with one another in an mTOR complex 3 (mTORC3); and

b) detecting said polynucleotide encoding the mTOR polypeptide or an active variant or fragment thereof and the polynucleotide encoding the TEL2 polypeptide or an active variant or fragment thereof.

28. A method for detecting an mTOR complex 3 (mTORC3), said method comprising:

a) contacting a sample with the antibody of any one of claims 6-12; and

b) detecting a complex comprising the mTORC3 and the antibody; thereby detecting said mTORC3.

29. A method for identifying an mTOR complex 3 (mTORC3) binding agent,

wherein the method comprises the steps of:

a) contacting the mTORC3 or a cell comprising the mTORC3 with a test compound; and

b) detecting a complex comprising the mTORC3 and the test compound.

30. The method of embodiment 29, wherein said method further comprises assaying the kinase activity of the mTORC3 to thereby determine if said test compound modulates the activity of the mTORC3 complex.

31. The method of embodiment 29 or 30, wherein said method further comprises contacting at least one of an mTORC1, an mTORC2, a cell comprising an mTORC1, and a cell comprising an mTORC2, and assaying for a complex comprising the mTORC1 or mTORC2 and the test compound, thereby determining if said test compound specifically binds to the mTORC3 complex.

32. The method of any one of embodiments 29-31, wherein said method is a cell-free method.

33. A method for screening for an mTOR complex 3 (mTORC3) antagonist, wherein said method comprises contacting mTORC3 with a test compound and assaying the kinase activity of the mTORC3 to thereby identify a compound that reduces the activity of the mTORC3.

34. The method of embodiment 33, wherein said method further comprises contacting at least one of an mTORC1, an mTORC2, a cell comprising an mTORC1, and a cell comprising an mTORC2, and assaying the kinase activity of the mTORC1 or mTORC2, thereby determining if said mTORC3 antagonist specifically reduces the activity of the mTORC3 complex.

35. The method of any one of embodiments 29-34, wherein said test compound comprises an antibody.

36. The method of any one of embodiments 29-34, wherein said test compound comprises a small molecule.

37. A method for reducing cell growth or cell survival, said method comprising contacting a cell expressing an mTOR complex 3 (mTORC3) with a specific mTORC3 antagonist.

38. The method of embodiment 37, wherein said specific mTORC3 complex antagonist comprises an antibody.

39. The method of embodiment 37, wherein said specific mTORC3 complex antagonist comprises a small molecule.

40. A method for treating or preventing a cancer in a subject in need thereof, wherein said method comprises administering to the subject a therapeutically effective amount of a specific mTORC3 complex antagonist.

41. The method of embodiment 40, wherein said specific mTORC3 complex antagonist comprises an antibody.

42. The method of embodiment 40, wherein said specific mTORC3 complex antagonist comprises a small molecule.

43. A method for diagnosing a cancer in a subject or determining the severity of a cancer in a subject, wherein said method comprises the steps of:

a) evaluating the level of an mTOR complex 3 (mTORC3) in a biological sample from said subject;

b) comparing the level of said mTORC3 in the biological sample of said subject to a control; and

c) diagnosing said cancer in said subject, wherein the level of said mTORC3 in the biological sample of said subject is relatively higher than the control; or determining the cancer of said subject is more severe than the control, wherein the level of mTORC3 in the biological sample of said subject is relatively higher than the control.

44. The method of embodiment 43, wherein said evaluating the level of mTORC3 in a sample of said subject comprises detecting the level of mTORC3 with an antibody of any one of embodiments 6-12 or a mixture of a first and a second antibody of embodiments 13-15.

45. The method of embodiment 43 or 44, wherein said method further comprises administering to the subject a therapeutically effective amount of a specific mTORC3 complex antagonist.

46. The method of any one of embodiments 40-45, wherein said cancer comprises a solid tumor cancer.

47. The method of any one of embodiments 40-45, wherein said cancer comprises a pediatric cancer.

48. The method of any one of embodiments 40-45, wherein said cancer is selected from the group consisting of acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, nephroblastoma (Wilm's tumor), hepatocellular carcinoma, esophageal carcinoma, liposarcoma, bladder cancer, gastric cancer, myxofibrosarcoma, colon cancer, kidney cancer, histiosarcoma, ovarian cancer, endometrial carcinoma, lung cancer, and breast cancer.

49. A method for treating or preventing a non-B cell cancer in a subject in need thereof, wherein said method comprises administering to the subject a therapeutically effective amount of a specific TEL2 antagonist, wherein said non-B cell cancer is selected from the group consisting of ependymoma, Ewing's sarcoma, glioblastoma, medulloblastoma, neuroblastoma, osteosarcoma, rhabdomyosarcoma, rhabdoid cancer, nephroblastoma (Wilm's tumor), esophageal carcinoma, liposarcoma, bladder cancer, gastric cancer, myxofibrosarcoma, colon cancer, kidney cancer, histiosarcoma, ovarian cancer, endometrial carcinoma, lung cancer, and breast cancer.

50. A method for diagnosing a non-B cell cancer in a subject or determining the severity of a non-B cell cancer in a subject, wherein said method comprises the steps of:

a) evaluating the expression of TEL2 in a biological sample from said subject;

b) comparing the expression of TEL2 in said biological sample of said subject with a control; and

c) diagnosing said non-B cell cancer in said subject, wherein the expression level of TEL2 in the biological sample of said subject is relatively higher than the control; or determining the non-B cell cancer of said subject is more severe than the control, wherein the expression level of TEL2 in the sample of said subject is relatively higher than the control, wherein said non-B cell cancer is selected from the group consisting of ependymoma, Ewing's sarcoma, glioblastoma, medulloblastoma, neuroblastoma, osteosarcoma, rhabdomyosarcoma, rhabdoid cancer, nephroblastoma (Wilm's tumor), esophageal carcinoma, liposarcoma, bladder cancer, gastric cancer, myxofibrosarcoma, colon cancer, kidney cancer, histiosarcoma, ovarian cancer, endometrial carcinoma, lung cancer, and breast cancer.

51. The method of embodiment 50, wherein said method further comprises administering to the subject a therapeutically effective amount of a specific mTORC3 complex antagonist or a specific TEL2 antagonist.

52. A method for treating an Epstein-Barr virus infection in a subject in need thereof, wherein said method comprises administering a therapeutically effective amount of a specific mTORC3 complex antagonist.

53. A non-human transgenic animal having stably incorporated into its genome a polynucleotide that encodes a TEL2 polypeptide or a biologically active variant or fragment thereof, wherein said polynucleotide is heterologous to the genome.

54. The non-human transgenic animal of embodiment 53, wherein said polynucleotide encodes the TEL2 polypeptide of SEQ ID NO: 4, a biologically active fragment thereof, or a biologically active variant thereof having at least 80% sequence identity to the TEL2 polypeptide of SEQ ID NO: 4.

55. The non-human transgenic animal of embodiment 53 or 54, wherein said non-human transgenic animal comprises a single copy of the stably incorporated polynucleotide.

56. The non-human transgenic animal of any one of embodiments 53-55, wherein said polynucleotide encoding the TEL2 polypeptide further comprises a TEL2 promoter.

57. The non-human transgenic animal of any one of embodiments 53-56, wherein said non-human transgenic animal is heterozygous for a p53 mutation that inhibits p53 activity.

58. The non-human transgenic animal of any one of embodiments 53-57, wherein said non-human transgenic animal is a rodent.

These and other aspects of the invention are disclosed in more detail in the description of the invention given below.

### BRIEF DESCRIPTION OF THE FIGURES

FIG. 1 provides immunoblots of cell lysates of wild type (left panel) and Arf^{-/-} (right panel) mouse pre-B cells expressing GFP (vector) or TEL2 and GFP (TEL2) were probed for the presence of mTOR, phospho-p70S6K^{Thr389}, p70S6K, phospho-Akt^{Ser473}, AKT, phospho-NDRG1^{Thr346}, NDRG1, phospho-S6^{Ser235/236}, S6, phospho-4E-BP1^{T37/46}, phospho-4E-BP1^{Ser65}, phospho-4E-BP1^{Thr70}, 4E-BP1 and TEL2. Tubulin was used as a loading control.

FIGS. 2A and 2B provide immunoblots of whole cell lysates of mouse Arf^{-/-} pre-B cells expressing vector (V) or TEL2 (T2) (FIG. 2A), Karpas-299 cells, K562 cells and OS-17 cells (FIG. 2B) that were immunoprecipiated with mTOR (mTOR IP) or TEL antibodies (TEL2 IP). The immunoprecipitated material was immunoblotted for the presence of mTOR, Rictor, Raptor, mSIN1, mLST8 and TEL2. The Raptor antibody did not give any signal in mTOR IPs in mouse Arf^{-/-} pre-B cells, due to very low amounts of Raptor in these cells. IgG indicates immunoprecipitation with a non-relevant antibody.

FIG. 2C provides immunoblots of purified HEK293T cell-derived mTOR and TEL2 proteins that were co-incubated for 8 or 24 hours or were co-incubated for these time intervals with purified recombinant RUVBL2. After immunoprecipitation with mTOR (mTOR IP) or TEL2 (TEL2 IP) antibodies, the material was immunoblotted for the presence of mTOR, TEL2 and RUVBL2. Input shows the purified mTOR, TEL2 and RUVBL2 preparations.

FIG. 3A provides immunoblots of sub-cellular fractions (C=cytoplasm, M=membrane, N=nuclear) of Karpas-299 cells that were immunoprecipitated with mTOR (mTOR IP) or TEL2 (TEL2 IP) antibodies, followed by immunoblotting using a mTOR (mTOR), TEL2 (TEL2), Rictor (Rictor) or Raptor (Raptor) antibody. Only cytoplasmic mTOR and TEL2 co-immunoprecipitate. The immunoblot on the right shows the purity of the sub-cellular fractions using markers specific for the cytoplasm (Tubulin), membrane (LAMP1) and nucleus (DEK).

FIG. 3B provides immunoblots of lysates of Karpas-299 cells subjected to Superose-6 FPLC gel filtration, followed by immunoprecipitation of the fractions (Fxn) with a TEL2 antibody and immunoblotting for the presence of mTOR and p-4E-BP 1^{37/46}. Numbers above the lanes indicate column fractions. The graph underneath the blot shows the elution profile of a mixture of different molecular weight markers (1.7-670 kDa.) on this column, which has been used to roughly estimate the molecular weight of the column fractions. Column fraction 9 represents the 8^{th} ml of column elution volume as indicated by the bent arrow. mTORC3 is larger than 1.5 mDa.

FIG. 3C provides immunoblots of lysates from xenograft tumors BT-28 and BT-39 next to lysates of Karpas-299 (K-299) that were immunoprecipitated with non-relevant IgG (IgG control) or TEL2 (TEL2 IP) antibodies followed by immunoblotting with an mTOR (mTOR), TEL2 (TEL2) antibody, or p-4E-BP 1^{37/46} antibody.

FIG. 4 demonstrates that the mTORC3 complex has in vitro kinase activity. FIG. 4A provides an immunoprecipitation/Western blot of Karpas-299 cell lysates showing the total amount of mTOR present in these cells (mTOR IP), the amount present in mTORC3 (TEL2 IP), in mTORC1 (Raptor IP) and mTORC2 (Rictor IP). Most mTOR is bound to mTORC2, much less to mTORC3, and the least to mTORC1. Rictor and Raptor antibodies bring down mTOR, but not TEL2. TEL2 antibody brings down mTOR, but not Raptor or Rictor. IgG indicates the immunoprecipitation with non-relevant IgG. FIGS. 4B-4C provide Western blots of recombinant 4E-BP1 and AKT protein, respectively, that was incubated with the Karpas-299 immunoprecipitated material as shown in FIG. 4A in the presence of ATP. The blots show the amount of p-4E-BP1^{Thr37/46} and p-AKT^{Ser473} phosphorylation by mTORC1+mTORC2+mTORC3 (mTOR IP), mTORC3 alone (TEL2 IP), mTORC1 alone (Raptor IP), and mTORC2 alone (Rictor IP). IgG shows the level of background phosphorylation of the 4E-BP1 and AKT substrates by non-relevant IgG immunoprecipitated material. FIG. 4D shows the results of an IP/kinase experiment using endogenous TEL2-expressing Karpas-299 cell lysates immunoprecipitated with mTOR (mTOR IP) or TEL2 (TEL2-IP) antibodies and incubated with recombinant 4E-BP1 in the presence of γ³²P-labeled ATP in the absence of inhibitor (no inhib), or in the presence of FKBP12/Rapamycin (Rapa, 37 μg/ml/20 μM), or OSI-27 (OSI-27, 10 μM). The negative control shows labeling of 4E-BP1 in the kinase assay after immunoprecipitation with non-relevant IgG (IgG). The histogram underneath the autoradiogram represents the relative intensities of the ³²P-labeled bands normalized to the IgG control.

FIG. 5 shows that mTORC3 kinase activity is insensitive to Rapamycin but sensitive to AZD-8055 and OSI-27 in cultured cells. FIG. 5A provides a graph presenting cell density as a percent control of Karpas-299 cells. Logarithmically growing Karpas-299 cells were treated for three population doublings with increasing amounts (0.1, 0.3, 1, 3, 10, 30, 100, 300, 1000, 3000, 10,000 ng/ml) of Rapamycin, AZD-8055 or OSI-27. Cell densities were plotted as the percentage of cells treated with vehicle. FIG. 5B provides a graph presenting cell density as a percent control of mouse pre-B cells transduced with MSCV-IRES-GFP (vector) or MSCV-TEL2-IRES-GFP (TEL2) and treated with AZD-8055 or Rapamycin. Cell densities were plotted as the percentage of cells treated with vehicle. FIG. 5C provides m-TOR IP/immunoblots of lysates of the Rapamycin-treated Karpas-299 cells probed for the presence of Rictor, mSIN1, mLST8 and TEL2. FIG. 5D provides immunoblots of the same Rapamycin-treated fractions of FIG. 5C probed for the presence of p-mTOR^{Ser2448}, p-AKT^{Ser473}, p-AKT^{Thr308}, p-P70S6K^{Thr389}, p-S6^{Ser235/236}, p-4E-BP1^{Thr37/46} and for the autophagosome marker LC3B I/II. FIG. 5E shows immunoblots of Karpas-299 lysates of AZD-8055-treated fractions probed for the presence of mTOR, p-AKT^{Ser473}, p-AKt^{Thr308}, AKT, p-S6^{Ser235/236}, S6, p-4E-BP1^{Thr37/46}, KI-67 and the autophagosome marker LC3B I/II. FIG. 5F shows immunoblots of mouse Arf^{-/-} pre-B cells transduced with MSCV-TEL2-IRES-GFP retrovirus treated with AZD-8055 probed for the presence of mTOR, p-AKT^{Ser473}, AKT, p-S6^{Ser235/236}, S6, p-4E-BP1^{Thr37/46}, KI-67, TEL2, and the autophagosome marker LC3B I/II. FIG. 5G shows immunoblots of lysates of Karpas-299 cells transduced with lentiviral vectors expressing scrambled shRNA, Raptor shRNA or Rictor shRNA, probed for the presence of mTOR, Rictor, Raptor, p-P70S6K^{Thr389}, P70S6K, p-AKT⁴⁷³, AKT, p-NDRG1^{Thr346}, p-S6^{Ser235/236}, S6, p-4E-BP1^{Thr37/46}, 4E-BP1, mLST8, TEL2, p-ERK1/2^{Thr202/Tyr204}, and tubulin. In the Raptor knockdown cells (no mTORC1), there is still phosphorylation of the mTORC1-specific substrates p-P70S6K^{Thr389}, S6^{Ser235/236}, p-4E-BP1^{Thr37/46}, while in the Rictor knockdown (no mTORC2) there is still phosphorylation of the mTORC2-specific substrates p-AKT^{Ser473} and p-NDRG1^{Thr346}, confirming Raptor and Rictor-independent mTOR activity of mTORC3.

FIG. 6 demonstrates that knockdown of TEL2 in OS-17 osteosarcoma cells inhibits proliferation. FIG. 6A provides a depiction of the pCL20-TRIPZ lentiviral construct used for doxycycline-inducible expression of shRNA. LTR=long terminal repeat; t^{o}p=tetracycline operator/CMV minimal promoter; RFP=red fluorescent protein; sh=TEL2shRNA; Ubqp=ubiquitin promoter; tTA=TetNP16 transactivator; Ac=mouse actin promoter; GFP=green fluorescent protein; ΔL=deleted LTR; rBGpA=rabbit b-globin poly(A) sequence. FIG. 6B provides the results of an experiment in which OS-17 cells were transduced with a non-targeting sh-RNA retroviral vector (NT) or a TEL2-shRNA retroviral vector (TEL2). After transduction, GFP⁺ cells were sorted (GFP) and induced with doxycycline for 72 hours. Both GFP⁺ (GFP) and GFP⁺/RFP⁺ (RFP) cells were sorted from the induced cultures, lysed and immunoprecipitated with TEL2 antibody and immunoblotted for TEL2 and mTOR. The histogram beneath the blot shows the level of TEL2 knockdown in the TEL2-shRNA GFP⁺/RFP⁺ cells. Lysates from the sorted cells were immunoblotted for p-AKT^{Ser473}, total AKT, p-4EBP1^{Thr37/46} and total 4EBP1. Tubulin was used as a loading control.

FIG. 7A shows TEL2 expression (brown staining) in human pancreas, colon and stomach tissue sections (human) and in sections of the same tissues of a TEL2-BAC+/− transgenic mouse (BAC TG) and a wild type mouse (WT mouse). There is no staining in tissues of the wild type mouse. FIG. 7B shows the difference in survival between mice carrying a single copy integration of a TEL2-BAC transgene on a heterozygous p53 knockout background (TEL2^{TG}/p53^{+/-}) and mice that are heterozygous for the p53 knockout mutation alone (P53^{+/-}). Tumors in TEL2-BAC^{+/-}/p53^{+/-} mice start to appear 4-fold earlier and at a much higher penetrance than in p53^{+/-} mice. FIG. 7C, left panel (H&E), shows the hematoxylin and eosin staining of an osteosarcoma that developed in a TEL2-BAC^{+/-}/p53^{+/-} mouse. The flanking 3 panels show adjacent sections stained with TEL2 antibody (TEL2), TEL2 antibody in the presence of excess peptide against which the antibody was raised (TEL2+peptide), and p-4E-BP1^{Thr37/46} antibody (p-4E-BP1^{Thr37/46}), respectively. The faster proliferating outer edge of the tumor stains stronger with TEL2 (brown stain) and p-4E-BP1^{Thr37/46} antibody (brown stain), indicating higher expression of the TEL2 transgene, resulting in a higher level of p-4E-BP1^{Thr37/46} phosphorylation due to increased mTORC3 activity.

### DETAILED DESCRIPTION OF THE INVENTION

### I. Compositions

A novel mammalian target of rapamycin (mTOR)-containing protein complex, mTOR complex 3 (mTORC3) is provided. As demonstrated herein, the mTORC3 comprises mTOR and translocation Ets leukemia 2/ets variant 7 (TEL2/ETV7).

As used herein, the “mTOR complex 3” or “mTORC3” refers to any molecular complex comprising at least one mTOR polypeptide or biologically active variant or fragment thereof and at least one TEL2 polypeptide or biologically active variant or fragment thereof, wherein the complex has or is capable of being activated to have at least one of the following biological activities: (1) the ability to phosphorylate at least one mTORC1 substrate (e.g., 4EBP1, p70S6K) and at least one mTORC2 substrate (e.g., Akt, protein kinase Cα, SGK-1); and (2) stimulating cell growth, proliferation, or survival. In some embodiments, the mTORC3 further comprises 4E-BP1. As used herein, the terms “4E-BP1”, “eukaryotic translation initiation factor 4E-binding protein 1”, and “EIF4E-BP1” can be used interchangeably and refer to a protein that interacts directly with eukaryotic translation initiation factor 4E (eIF4E) and represses cap-dependent translation by inhibiting the assembly of the multisubunit complex that recruits 40S ribosomal subunits to the 5′ end of mRNAs.

4E-BP1 polynucleotides and polypeptides are known in the art (Pause et al. (1994) Nature 371:762-767, which is herein incorporated by reference in its entirety). Non-limiting examples of 4E-BP1 polynucleotides and polypeptides comprise the human 4E-BP1 polynucleotide as set forth in SEQ ID NO:5 (nucleotides 73-429 of GenBank Accession No. NM_{—}004095) and the encoded human 4E-BP1 polypeptide (Accession No. NP_{—}004086) as set forth in SEQ ID NO: 6. In some of those embodiments wherein mTORC3 further comprises 4E-BP1, 4E-BP1 is phosphorylated on the threonine residues corresponding to positions 37 and 46 of SEQ ID NO: 6 (referred to herein as 4E-BP1^{Thr37/46}).

In some embodiments, the mTORC3 has a molecular weight greater than 1.5 mDa, including but not limited to about 1.6 mDa, 1.7 mDa, 1.8 mDa, 1.9 mDa, 2.0 mDa, or greater.

Unlike mTORC 1 and mTORC2, mTORC3 is insensitive to inhibition by rapamycin. Therefore, the mTOR complex 3 is stable even in the presence of relatively high concentrations of rapamycin (or an analog thereof). In some embodiments, the mTOR complex 3 is stable in the presence of 1 ng/ml or greater of rapamaycin or an analog thereof, including but not limited to about 1 ng/ml, 2 ng/ml, 5 ng/ml, 10 ng/ml, 50 ng/ml, 100 ng/ml, 150 ng/ml, 200 ng/ml, 250 ng/ml, 300 ng/ml, 500 ng/ml or greater of rapamycin or an analog thereof.

The most well-characterized mTORC1 substrates are 4EBP1 and p70S6K. mTORC1 phosphorylates 4EBP1 at threonine 37 and 46 (Thr37/46) and p70S6K at threonine 389 (Thr389). mTORC2 phosphorylates Akt at Ser-473, protein kinase Cα (PKCα) at Ser-657, and SGK-1 at Ser-422. In some embodiments, an active mTORC3 has a kinase activity for an mTORC1 substrate and/or an mTORC2 substrate that is at least 2-fold higher than that of mTORC1 and/or mTORC2, including but not limited to, about 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 50-fold, 100-fold, or higher than that of mTORC1 or mTORC2. The kinase activity of an mTOR-comprising complex can be measured using methods known in the art (see, for example, Chiang and Abraham (2004) Methods Mol Biol 281:125-141), including but not limited to, those described elsewhere herein (see Experimental).

In some embodiments, the mTOR and TEL2 polypeptides are associated with one another directly (e.g., through covalent or non-covalent interactions). In particular embodiments, the mTOR complex 3 does not comprise Rictor, Raptor, mLST8, or SIN1.

1. Polynucleotides and Polypeptides

The methods and compositions of the invention utilize various polynucleotides and polypeptides. As used herein, the term “polynucleotide” is intended to encompass a singular nucleic acid, as well as plural nucleic acids, and refers to a nucleic acid molecule or construct, e.g., messenger RNA (mRNA), plasmid DNA (pDNA), or short interfering RNA (siRNA). A polynucleotide can be single-stranded or double-stranded, linear or circular and can be comprised of DNA, RNA, or a combination thereof. A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. The “polynucleotide” can contain modified nucleic acids, such as phosphorothioate, phosphate, ring atom modified derivatives, and the like. The “polynucleotide” can be a naturally occurring polynucleotide (i.e., one existing in nature without human intervention), a recombinant polynucleotide (i.e., one existing only with human intervention), or a synthetically derived polynucleotide.

As used herein, the term “polypeptide” or “protein” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms.

An “isolated” or “purified” polynucleotide, protein, or protein complex is substantially or essentially free from components that normally accompany or interact with the polynucleotide, protein, or protein complex as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences that naturally flank the polynucleotide (i.e., sequences located at the 5′ and 3′ ends of the polynucleotide) in the genomic DNA of the organism from which the polynucleotide is derived. A protein or protein complex that is substantially free of cellular material includes preparations of protein having less than about 30%, 20%, 10%, 5%, or 1% (by dry weight) of contaminating protein. When the protein is recombinantly produced, optimally culture medium represents less than about 30%, 20%, 10%, 5%, or 1% (by dry weight) of chemical precursors or non-protein-of-interest chemicals.

For the purposes of the present invention, a “coding sequence for a polypeptide of interest” or “coding region for a polypeptide of interest” refers to the polynucleotide sequence that encodes that polypeptide. As used herein, the terms “encoding” or “encoded” when used in the context of a specified nucleic acid mean that the nucleic acid comprises the requisite information to direct translation of the nucleotide sequence into a specified polypeptide. The information by which a polypeptide is encoded is specified by the use of codons. The “coding region” or “coding sequence” is the portion of the nucleic acid that consists of codons that can be translated into amino acids. Although a “stop codon” or “translational termination codon” (TAG, TGA, or TAA) is not translated into an amino acid, it can be considered to be part of a coding region. Likewise, a transcription initiation codon (ATG) may or may not be considered to be part of a coding region. Any sequences flanking the coding region, however, for example, promoters, ribosome binding sites, transcriptional terminators, introns, and the like, are not considered to be part of the coding region.

i. mTOR Polynucleotides and Polypeptides

The mTOR complex 3 (mTORC3) comprises mTOR and TEL2. As used herein, the terms “mammalian target of rapamycin”, “mTOR”, “TOR”, “FK506 binding protein 12-rapamycin associated protein 1”, and “FRAP1” can be used interchangeably and refer to a member of the phosphoinositide-3-kinase-related kinase (PI3K-related kinase or PIKK) family that regulates a variety of cellular processes, including growth, proliferation, survival, motility, protein synthesis, and transcription. PI3K-related kinases comprise a carboxyl terminal kinase domain having significant sequence homology to the phosphoinositide 3-kinase (PI3K) catalytic domain; however, unlike PI3K, which is a lipid kinase, the PI3K-related kinases function as serine-threonine kinases. mTOR is also known as FK506 binding protein 12-rapamycin associated protein 1 (FRAP1).

mTOR had been reported to reside in two physically and functionally distinct signaling complexes, mTORC 1 and mTORC2. Each complex has a unique subunit composition and unique substrates. The presently disclosed subject matter describes a third, novel mTOR-comprising complex, mTORC3, capable of phosphorylating both mTORC1- and mTORC2-specific substrates. mTOR polynucleotides and polypeptides are known in the art. Non-limiting examples of mTOR polynucleotides and polypeptides comprise the human mTOR polynucleotide as set forth in SEQ ID NO:1 that can be found in GenBank Accession No. NM_{—}004958 and the encoded human mTOR polypeptide (Accession No. NP_{—}004949) as set forth in SEQ ID NO: 2.

mTOR polypeptides comprise a variety of conserved structural motifs. For ease of reference, such motifs will be discussed as they relate to the human mTOR which is set forth in SEQ ID NO:2. mTOR polypeptides comprise two tandem arrays of HEAT (Huntington, Elongation factor 3A, A subunit of PP2A, and TOR1) repeats (with HEAT repeats from about amino acid residues 16 to 53, 650 to 688, 859 to 897, 988 to 1025, 1069 to 1106, 1109 to 1148, 1150 to 1186 of SEQ ID NO:2), which likely mediate protein-protein interactions; followed by a FAT (FRAP, ATM, and TRRAP) domain (from about amino acid residues 1382 to 1982 of SEQ ID NO:2), the function of which is unknown, but it is relatively conserved in the FRAP, ATM, and TRRAP PIKK family members. The FAT domain is followed by the phosphoinositide-3-kinase-related catalytic domain (from about amino acid residues 2182 to 2516 of SEQ ID NO: 2); and a FAT-C domain (from about amino acid residues 2517 to 2549 of SEQ ID NO: 2). Together, the FAT and FAT-C domain might contribute to the active conformation of the intervening kinase domain. Phosphorylation of mTOR can occur at threonine 2446 (Thr2446) of SEQ ID NO: 2, which has been reported to be phosphorylated by the Akt kinase (Sekulié et al. (2000) Cancer Res 60(13):3504-3513); at serine 2448 (Ser2448), which is phosphorylated by p70S6K (Chiang and Abraham (2005) J Biol Chem 280(27):25485-25490; Copp, Manning, and Hunter (2009) Cancer Res 69:1821-1827); and at serine 2481 (Ser2481) of SEQ ID NO: 2, an mTOR autophosphorylation site (Peterson et al. (2000) J Biol Chem 275(10):7416-7423).

It is recognized that biologically active variants and fragments of the mTOR polypeptide can be employed in the various methods and compositions of the invention. Such active variants and fragments will retain the ability to associate with TEL2 in the mTOR complex 3 (mTORC3) and in some embodiments, will retain a functional catalytic domain. Methods to assay for kinase activity or (direct or indirect) binding to TEL2 are known and are described elsewhere herein (see Experimental).

Thus, in some embodiments, the mTOR polypeptide used in the methods and compositions of the invention comprises the amino acid sequence as shown in SEQ ID NO:2 or a biologically active variant or fragment thereof. Some embodiments of the methods and compositions utilize mTOR polynucleotides comprising a nucleotide sequence encoding an mTOR polypeptide, and in some of these embodiments, the polynucleotide has the nucleotide sequence set forth in SEQ ID NO:1 or a biologically active variant or fragment thereof.

ii. TEL2 Polynucleotides and Polypeptides

As used herein, the terms “TEL2”, “translocation Ets leukemia 2”, “Ets variant gene 7”, “ETV7”, and “TEL2/ETV7” can be used interchangeably and refer to a member of the ETS (E26-transformation specific) transcription factor family that can homodimerize or heterodimerize with TEL1 and possibly other Ets family members, displays transcriptional repression activity, and has now been shown herein to be a subunit of the mTORC3 complex. TEL2 is predominantly expressed in human hematopoietic tissues both during development and adult life (Potter et al. (2000) Blood 95:3341-3348). TEL2 self-associates via its PNT (pointed) domain but can also form heterodimers with TEL1 (Potter et al. (2000) Blood 95:3341-3348). Despite their similarity in sequence and structure, TEL1 and TEL2 show opposite biological effects. For example, TEL1 suppresses Ras-induced transformation of NIH3T3 fibroblasts in vitro (Van Rompaey et al. (1999) Neoplasia 1:526-536), while TEL2 promotes it (Kawagoe et al. (2004) Cancer Res 64:6091-6100). Forced expression of TEL2, but not TEL1, inhibits vitamin-D3-induced differentiation of U937 cells (Kawagoe et al. (2004) Cancer Res 64:6091-6100). TEL2 inhibits apoptosis in murine bone marrow and pre-B cells cultured in vitro and cooperates with Myc in murine B-lymphomagenesis (Cardone et al. (2005) Mol Cell Biol 25:2395-2405). TEL2 overexpression also accelerates cell cycle traverse of mouse pre-B cells (Cardone et al. (2005) Mol Cell Biol 25:2395-2405).

TEL2 is conserved among vertebrate species but the gene underwent deletion in rodents possibly at or after the split with the lagomorpha, because the gene is present in rabbit. (Ensemble genetree, which can be found on the world wide web at uswest.ensembl.org/Homo_sapiens/Gene/Compara_Tree?collapse=1895549%2C1895902; db=core; g=ENSG00000010030; r=6:36322419-36356164). Thus, TEL2 polynucleotides and polypeptides are known in the art (Potter et al. (2000) Blood 95(11):3341-3348; Poirel et al. (2000) Oncogene 19:4802-4806; Gu et al. (2001) J Biol Chem 276(12):9421-9436, each of which are herein incorporated by reference in its entirety). Non-limiting examples of TEL2 polynucleotides and polypeptides include the human TEL2 polynucleotide set forth in SEQ ID NO: 3 and which can be found in GenBank Accession No. NM_{—}016135, and the human TEL2 polypeptide set forth in SEQ ID NO: 4 (Accession No. NP_{—}057219).

The TEL2 polypeptide comprises a variety of conserved structural motifs. For ease of reference, such motifs will be discussed as they relate to the human TEL2 which is set forth in SEQ ID NO:4. TEL2 polypeptides comprise a sterile alpha motif/pointed (SAM/PNT) domain (from about amino acid residues 33 to 117 of SEQ ID NO:4, which is believed to mediate protein/protein interactions; and an Ets domain (from about amino acid residues 224 to 305 of SEQ ID NO: 4), which comprises a DNA binding domain. TEL2 has a putative ATM/ATR/DNA-PK kinase phosphorylation site at serine 324 (Ser324) of SEQ ID NO: 4).

It is recognized that biologically active variants and fragments of the TEL2 polypeptide can be employed in the various methods and compositions of the invention. Such active variants and fragments will continue to retain the ability to associate with mTOR in an mTOR complex 3 (mTORC). TEL2 mutants missing the PNT or Ets domain are inactive in transformation or growth stimulation of mouse pre-B cells (Cardone et al. (2005) Mol Cell Biol 25:2395) and do not inhibit chemically-induced differentiation of U937 cells (Kawagoe et al. (2004) Cancer Res 64:6091-6100). Thus, in some embodiments, the TEL2 polypeptide variant or fragment comprises the SAM/PNT domain. In other embodiments, the TEL2 polypeptide variant or fragment comprises the Ets domain and retains the ability to bind to DNA. In still other embodiments, the TEL2 polypeptide variant or fragment comprises both the SAM/PNT domain and the Ets domain. Methods to assay for binding to mTOR or association with the mTORC3 complex are known and described elsewhere herein (see Experimental). Variants and fragments of TEL2 polypeptides and polynucleotides are known in the art including, but not limited to the alternatively spliced variants described by Gu et al. (2001) J Biol Chem 276(12):9421-9436.

Thus, in one embodiment, the TEL2 polypeptide used in the methods and compositions of the invention comprises the amino acid sequence as shown in SEQ ID NO:4 or a biologically active variant or fragment thereof. Some embodiments of the methods and compositions utilize TEL2 polynucleotides comprising the nucleotide sequence encoding a TEL2 polypeptide, and in some of these embodiments, the polynucleotide has the nucleotide sequence set forth in SEQ ID NO:3 or a biologically active variant or fragment thereof.

iii. Fragments and Variants

Fragments and variants of the polynucleotides encoding the mTOR and TEL2 polypeptides can be employed in the various methods and compositions of the invention.

By “fragment” is intended a portion of the polynucleotide and hence the protein encoded thereby or a portion of the polypeptide. Fragments of a polynucleotide may encode protein fragments that retain the biological activity of the native protein and hence have the ability to associate with other mTORC3 subunits. A fragment of a polynucleotide that encodes a biologically active portion of an mTOR or TEL2 polypeptide will encode at least 15, 25, 30, 50, 100, 150, 200, 250, or 300 contiguous amino acids, or up to the total number of amino acids present in a full-length mTOR or TEL2 polypeptide.

A biologically active portion of an mTOR or TEL2 polypeptide can be prepared by isolating a portion of one of the polynucleotides encoding the portion of the mTOR or TEL2 polypeptide and expressing the encoded portion of the polypeptide (e.g., by recombinant expression in vitro), and assessing the activity of the portion of the mTOR or TEL2 polypeptide. Polynucleotides that encode fragments of an mTOR or TEL2 polypeptide can comprise nucleotide sequences comprising at least 15, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length mTOR or TEL2 nucleotide sequence disclosed herein.

“Variant” sequences have a high degree of sequence similarity. For polynucleotides, conservative variants include those sequences that, because of the degeneracy of the genetic code, encode the amino acid sequence of one of the mTOR or TEL2 polypeptides. Variants such as these can be identified with the use of well-known molecular biology techniques, such as, for example, polymerase chain reaction (PCR) and hybridization techniques. Variant polynucleotides also include synthetically derived nucleotide sequences, such as those generated, for example, by using site-directed mutagenesis, but which still encode an mTOR or a TEL2 polypeptide. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide can also be evaluated by comparison of the percent sequence identity between the polypeptide encoded by a variant polynucleotide and the polypeptide encoded by the reference polynucleotide. Thus, variants include, for example, isolated polynucleotides that encode a polypeptide with a given percent sequence identity to the mTOR and TEL2 polypeptides set forth herein. Percent sequence identity between any two polypeptides can be calculated using sequence alignment programs and parameters described herein. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

By “variant” polypeptide is intended a polypeptide derived from the native polypeptide by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. Variant mTOR and TEL2 polypeptides can be biologically active, that is they continue to possess the desired biological activity of the native polypeptide, that is, the ability to associate with other mTORC3 subunits. Such variants may result from, for example, genetic polymorphism or from human manipulation. Biologically active variants of an mTOR or TEL2 polypeptide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native polypeptide as determined by sequence alignment programs and parameters described elsewhere herein. A biologically active variant of a polypeptide may differ from that polypeptide by as few as 1-15 amino acid residues, as few as 1-10, such as 6-10, as few as 5, as few as 4, 3, 2, or even 1 amino acid residue.

Polypeptides may be altered in various ways including amino acid substitutions, deletions, truncations, and insertions. Methods for such manipulations are generally known in the art. For example, amino acid sequence variants of the mTOR or TEL2 polypeptides can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the polypeptide of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Thus, the polynucleotides used in the invention can include the naturally occurring sequences, the “native” sequences, as well as mutant forms. Likewise, the polypeptides used in the methods of the invention encompass naturally occurring polypeptides as well as variations and modified forms thereof. Generally, the mutations made in the polynucleotide encoding the variant polypeptide should not place the sequence out of reading frame, and/or create complementary regions that could produce secondary mRNA structure. See, EP Patent Application Publication No. 75,444.

The deletions, insertions, and substitutions of the polypeptide sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Variant polynucleotides and polypeptides also encompass sequences and polypeptides derived from a mutagenic and recombinogenic procedure such as DNA shuffling. With such a procedure, one or more different mTOR or TEL2 coding sequences can be manipulated to create a new mTOR or TEL2 polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

2. TEL2 Antagonists

The presently disclosed subject matter provides for methods of reducing the expression or activity of TEL2 using TEL2 antagonists.

The term “TEL2 antagonist” refers to an agent which reduces, inhibits, or otherwise diminishes one or more of the biological activities of TEL2, which includes the ability to associate with mTORC3 subunits, the ability to bind to DNA, the ability to repress transcription, the ability to reduce apoptosis and increase cell survival, and the ability to enhance cell proliferation. Antagonism using the TEL2 antagonist does not necessarily indicate a total elimination of the TEL2 activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of TEL2 compared to an appropriate control.

By “specific antagonist” is intended an agent that reduces, inhibits, or otherwise diminishes the activity of a defined target. Thus, a TEL2 specific antagonist reduces the biological activity of TEL2 by a statistically significant amount (i.e., at least 5%, 10%, 20%, 30%, 40%, 50%, 60%, 70%, 80%, 90%, 100% or greater) and the agent does not modulate the biological activity of non-TEL2 polypeptides by a statistically significant amount (i.e., the activity of non-TEL2 polypeptides is modulated by less than 5%, 4%, 3%, 2% or 1%). One of skill will be aware of the proper controls that are needed to carry out such a determination. A TEL2 specific antagonist may or may not specifically bind to TEL2. TEL2 specific antagonists can include, but are not limited to, small molecules, antibodies, polypeptides, or polynucleotides.

The TEL2 antagonist used to reduce the expression or activity of TEL2 may comprise a TEL2 silencing element. As used herein, the term “silencing element” refers to a polynucleotide, which when expressed or introduced into a cell is capable of reducing or eliminating the level of expression of a target polynucleotide sequence or the polypeptide encoded thereby. In some embodiments, the silencing element can be operably linked to a promoter to allow expression of the silencing element in a cell.

In one embodiment, the silencing element encodes a zinc finger protein that binds to a TEL2 gene, resulting in reduced expression of the gene. In particular embodiments, the zinc finger protein binds to a regulatory region of a TEL2 gene. In other embodiments, the zinc finger protein binds to a messenger RNA encoding a TEL2 and prevents its translation. Methods of selecting sites for targeting by zinc finger proteins have been described, for example, in U.S. Pat. No. 6,453,242, which is herein incorporated by reference.

In some embodiments of the invention, the silencing element encodes an antibody that binds to a TEL2 polypeptide and inhibits its activity (e.g., prevents it from forming mTORC3). In another embodiment, the binding of the antibody results in increased turnover of the antibody-TEL2 complex. In other embodiments of the invention, the silencing element encodes a polypeptide that specifically inhibits the activity of a TEL2.

In some embodiments of the present invention, the activity of TEL2 is reduced or eliminated by disrupting a TEL2 gene. The TEL2 gene may be disrupted by any method known in the art. For example, in one embodiment, the gene is disrupted by transposon tagging. In another embodiment, the gene is disrupted by mutagenizing cells using random or targeted mutagenesis, and selecting for cells that have reduced TEL2 activity.

In one embodiment of the invention, transposon tagging is used to reduce or eliminate the activity of TEL2. Transposon tagging comprises inserting a transposon within an endogenous TEL2 gene to reduce or eliminate expression of the TEL2. In this embodiment, the expression of the TEL2 gene is reduced or eliminated by inserting a transposon within a regulatory region or coding region of the TEL2 gene. A transposon that is within an exon, intron, 5′ or 3′ untranslated sequence, a promoter, or any other regulatory sequence of a TEL2 gene may be used to reduce or eliminate the expression and/or activity of the encoded TEL2. In these embodiments, the silencing element comprises or encodes a targeted transposon that can insert within a TEL2 gene.

In other embodiments, the silencing element comprises a nucleotide sequence useful for site-directed mutagenesis via homologous recombination within a region of a TEL2 gene. Insertional mutations in gene exons usually result in null mutants. The invention encompasses additional methods for reducing or eliminating the activity or expression of TEL2, such as those that involve promoter-based silencing. See, for example, Mette et al. (2000) EMBO J. 19: 5194-5201; Sijen et al. (2001) Curr. Biol. 11: 436-440; Jones et al. (2001) Curr. Biol. 11: 747-757, each of which are herein incorporated by reference in its entirety.

The silencing element can comprise or encode an antisense oligonucleotide or an interfering RNA (RNAi). The term “interfering RNA” or “RNAi” refers to any RNA molecule which can enter an RNAi pathway and thereby reduce the expression of a target gene. The RNAi pathway features the Dicer nuclease enzyme and RNA-induced silencing complexes (RISC) that function to degrade or block the translation of a target mRNA. RNAi is distinct from antisense oligonucleotides that function through “antisense” mechanisms that typically involve inhibition of a target transcript by a single-stranded oligonucleotide through an RNase H-mediated pathway. See, Crooke (ed.) (2001) “Antisense Drug Technology: Principles, Strategies, and Applications” (1st ed), Marcel Dekker; ISBN: 0824705661; 1st edition.

As used herein, the term “gene” has its meaning as understood in the art. In general, a gene is taken to include gene regulatory sequences (e.g., promoters, enhancers, and the like) and/or intron sequences, in addition to coding sequences (open reading frames). It will further be appreciated that definitions of “gene” include references to nucleic acids that do not encode proteins but rather encode functional RNA molecules, or precursors thereof, such as microRNA or siRNA precursors, tRNAs, and the like.

As used herein, a “target gene” comprises any gene that one desires to decrease the level of expression. By “reduces” or “reducing” the expression level of a gene is intended to mean, the level of the encoded polynucleotide (i.e., target transcript) or the encoded polypeptide is significantly lower than the encoded polynucleotide level or encoded polypeptide level in an appropriate control which is not exposed to the silencing element. In particular embodiments, reducing the expression of a TEL2 gene results in less than 95%, less than 90%, less than 80%, less than 70%, less than 60%, less than 50%, less than 40%, less than 30%, less than 20%, less than 10%, or less than 5% of the level of the Tel2 transcript or the level of the TEL2 polypeptide in an appropriate control (e.g., the same cell or a similar cell at a similar stage in differentiation, same phenotype, same genotype. etc. prior to the introduction/expression of the silencing element). Methods to assay for the level of the RNA transcript, the level of the encoded polypeptide, or the activity of the polynucleotide or polypeptide are known in the art, and are described elsewhere herein.

The term “complementary” is used herein in accordance with its art-accepted meaning to refer to the capacity for precise pairing via hydrogen bonds (e.g., Watson-Crick base pairing or Hoogsteen base pairing) between two nucleosides, nucleotides or nucleic acids, and the like. For example, if a nucleotide at a certain position of a first nucleic acid is capable of stably hydrogen bonding with a nucleotide located opposite to that nucleotide in a second nucleic acid, when the nucleic acids are aligned in opposite 5′ to 3′ orientation (i.e., in anti-parallel orientation), then the nucleic acids are considered to be complementary at that position (where position may be defined relative to either end of either nucleic acid, generally with respect to a 5′ end). The nucleotides located opposite one another can be referred to as a “base pair.” A complementary base pair contains two complementary nucleotides, e.g., A and U, A and T, G and C, and the like, whereas a noncomplementary base pair contains two noncomplementary nucleotides (also referred to as a mismatch). Two polynucleotides are said to be complementary to each other when a sufficient number of corresponding positions in each molecule are occupied by nucleotides that hydrogen bond with each other, i.e., a sufficient number of base pairs are complementary.

The term “hybridize” as used herein refers to the interaction between two complementary nucleic acid sequences in which the two sequences remain associated with one another under appropriate conditions.

A silencing element can comprise the interfering RNA or antisense oligonucleotide, a precursor to the interfering RNA or antisense oligonucleotide, a template for the transcription of an interfering RNA or antisense oligonucleotide, or a template for the transcription of a precursor interfering RNA or antisense oligonucleotide, wherein the precursor is processed within the cell to produce an interfering RNA or antisense oligonucleotide. Thus, for example, a dsRNA silencing element includes a dsRNA molecule, a transcript or polyribonucleotide capable of forming a dsRNA, more than one transcript or polyribonucleotide capable of forming a dsRNA, a DNA encoding a dsRNA molecule, or a DNA encoding one strand of a dsRNA molecule. When the silencing element comprises a DNA molecule encoding an interfering RNA, it is recognized that the DNA can be transiently expressed in a cell or stably incorporated into the genome of the cell. Such methods are discussed in further detail elsewhere herein.

The silencing element can reduce or eliminate the expression level of a target gene by influencing the level of the target RNA transcript, by influencing translation of the target RNA transcript, or by influencing expression at the pre-transcriptional level (i.e., via the modulation of chromatin structure, methylation pattern, etc., to alter gene expression). See, for example, Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional interfering RNA that are capable of reducing or eliminating the expression of a target gene are known in the art and disclosed elsewhere herein.

Any region of a transcript from the target gene (i.e., target transcript) can be used to design a domain of the silencing element that shares sufficient sequence identity to allow for the silencing element to decrease the level of the polynucleotide or polypeptide encoded by the target gene. For instance, the silencing element can be designed to share sequence identity to the 5′ untranslated region of the target transcript, the 3′ untranslated region of the target transcript, exonic regions of the target transcript, intronic regions of the target transcript, and any combination thereof.

The ability of a silencing element to reduce the level of the target transcript can be assessed directly by measuring the amount of the target transcript using, for example, Northern blots, nuclease protection assays, reverse transcription (RT)-PCR, real-time RT-PCR, microarray analysis, and the like. Alternatively, the ability of the silencing element to reduce the level of the polypeptide encoded by the target gene and target transcript can be measured directly using a variety of affinity-based approaches (e.g., using a ligand or antibody that specifically binds to the target polypeptide) including, but not limited to, Western blots, immunoassays, ELISA, flow cytometry, protein microarrays, and the like. In still other methods, the ability of the silencing element to reduce the level of the target polypeptide encoded by the target gene can be assessed indirectly, e.g., by measuring a functional activity of the polypeptide encoded by the transcript or by measuring a signal produced by the polypeptide encoded by the transcript.

Those of ordinary skill in the art will readily appreciate that a silencing element can be prepared according to any available technique including, but not limited to, chemical synthesis, enzymatic or chemical cleavage in vivo or in vitro, template transcription in vivo or in vitro, or combinations of the foregoing.

Various types of silencing elements are discussed in further detail below.

In one embodiment, the silencing element comprises or encodes a double stranded RNA molecule. As used herein, a “double stranded RNA” or “dsRNA” refers to a polyribonucleotide structure formed either by a single self-complementary RNA molecule or a polyribonucleotide structure formed by the expression of least two distinct RNA strands. Accordingly, as used herein, the term “dsRNA” is meant to encompass other terms used to describe nucleic acid molecules that are capable of mediating RNA interference or gene silencing, including, for example, small RNA (sRNA), short-interfering RNA (siRNA), double-stranded RNA (dsRNA), micro-RNA (miRNA), hairpin RNA, short hairpin RNA (shRNA), and others. See, for example, Meister and Tuschl (2004) Nature 431:343-349 and Bonetta et al. (2004) Nature Methods 1:79-86.

In specific embodiments, at least one strand of the duplex or double-stranded region of the dsRNA shares sufficient sequence identity or sequence complementarity to the target gene to allow for the dsRNA to reduce the level of expression of the target gene. As used herein, the strand that is complementary to the target transcript is the “antisense strand,” and the strand homologous to the target transcript is the “sense strand.”

In one embodiment, the dsRNA comprises a hairpin RNA. A hairpin RNA comprises an RNA molecule that is capable of folding back onto itself to form a double stranded structure. Multiple structures can be employed as hairpin elements. For example, the hairpin RNA molecule that hybridizes with itself to form a hairpin structure can comprise a single-stranded loop region and a base-paired stem. The base-paired stem region can comprise a sense sequence corresponding to all or part of the target transcript and further comprises an antisense sequence that is fully or partially complementary to the sense sequence. Thus, the base-paired stem region of the silencing element can determine the specificity of the silencing. See, for example, Chuang and Meyerowitz (2000) Proc. Natl. Acad. Sci. USA 97:4985-4990, herein incorporated by reference. A transient assay for the efficiency of hpRNA constructs to silence gene expression in vivo has been described by Panstruga et al. (2003) Mol. Biol. Rep. 30:135-140, herein incorporated by reference.

A “short interfering RNA” or “siRNA” comprises an RNA duplex (double-stranded region) and can further comprise one or two single-stranded overhangs, e.g., 3′ or 5′ overhangs. The duplex can be approximately 19 base pairs (bp) long, although lengths between 17 and 29 nucleotides, including 17, 18, 19, 20, 21, 22, 23, 24, 25, 26, 27, 28, and 29 nucleotides, can be used. An siRNA can be formed from two RNA molecules that hybridize together or can alternatively be generated from a single RNA molecule that includes a self-hybridizing portion. The duplex portion of an siRNA can include one or more bulges containing one or more unpaired and/or mismatched nucleotides in one or both strands of the duplex or can contain one or more noncomplementary nucleotide pairs. One strand of an siRNA (referred to herein as the antisense strand) includes a portion that hybridizes with a target transcript. In certain embodiments, one strand of the siRNA (the antisense strand) is precisely complementary with a region of the target transcript over at least about 17 nucleotides, 18 nucleotides, 19 nucleotides, 20 nucleotides, 21 nucleotides, or more meaning that the siRNA antisense strand hybridizes to the target transcript without a single mismatch (i.e., without a single noncomplementary base pair) over that length. In other embodiments, one or more mismatches between the siRNA antisense strand and the targeted portion of the target transcript can exist. In embodiments in which perfect complementarity is not achieved, any mismatches between the siRNA antisense strand and the target transcript can be located at or near the 3′ end of the siRNA antisense strand. For example, in certain embodiments, nucleotides 1-9, 2-9, 2-10, and/or 1-10 of the antisense strand are perfectly complementary to the target.

Considerations for the design of effective siRNA molecules are discussed in McManus et al. (2002) Nature Reviews Genetics 3: 737-747 and in Dykxhoorn et al. (2003) Nature Reviews Molecular Cell Biology 4: 457-467. Such considerations include the base composition of the siRNA, the position of the portion of the target transcript that is complementary to the antisense strand of the siRNA relative to the 5′ and 3′ ends of the transcript, and the like. A variety of computer programs also are available to assist with selection of siRNA sequences, e.g., from Ambion (web site having URL www.ambion.com), at the web site having the URL www.sinc.sunysb.edu/Stu/shilin/rnai.html. Additional design considerations that also can be employed are described in Semizarov et al. Proc. Natl. Acad. Sci. 100: 6347-6352.

The term “short hairpin RNA” or “shRNA” refers to an RNA molecule comprising at least two complementary portions hybridized or capable of hybridizing to form a double-stranded (duplex) structure sufficiently long to mediate RNAi (generally between approximately 17 and 29 nucleotides in length, including 17, 18, 19, 20, 21, 22, 23, 24, 25, 26, 27, 28, and 29 nucleotides in length, and in some embodiments, typically at least 19 base pairs in length), and at least one single-stranded portion, typically between approximately 1 and 20 or 1 to 10 nucleotides in length that forms a loop connecting the two nucleotides that form the base pair at one end of the duplex portion. The duplex portion can, but does not require, one or more bulges consisting of one or more unpaired nucleotides. In specific embodiments, the shRNAs comprise a 3′ overhang. Thus, shRNAs are precursors of siRNAs and are, in general, similarly capable of inhibiting expression of a target transcript.

In particular, RNA molecules having a hairpin (stem-loop) structure can be processed intracellularly by Dicer to yield an siRNA structure referred to as short hairpin RNAs (shRNAs), which contain two complementary regions that hybridize to one another (self-hybridize) to form a double-stranded (duplex) region referred to as a stem, a single-stranded loop connecting the nucleotides that form the base pair at one end of the duplex, and optionally an overhang, e.g., a 3′ overhang. The stem can comprise about 19, 20, or 21 bp long, though shorter and longer stems (e.g., up to about 29 nt) also can be used. The loop can comprise about 1-20, including 1, 2, 3, 4, 5, 6, 7, 8, 9, 10, 11, 12, 13, 14, 15, 16, 17, 18, 19, 20 nt, about 4-10, or about 6-9 nt. The overhang, if present, can comprise approximately 1-20 nt or approximately 2-10 nt. The loop can be located at either the 5′ or 3′ end of the region that is complementary to the target transcript whose inhibition is desired (i.e., the antisense portion of the shRNA).

Although shRNAs contain a single RNA molecule that self-hybridizes, it will be appreciated that the resulting duplex structure can be considered to comprise sense and antisense strands or portions relative to the target mRNA and can thus be considered to be double-stranded. It will therefore be convenient herein to refer to sense and antisense strands, or sense and antisense portions, of an shRNA, where the antisense strand or portion is that segment of the molecule that forms or is capable of forming a duplex with and is complementary to the targeted portion of the target polynucleotide, and the sense strand or portion is that segment of the molecule that forms or is capable of forming a duplex with the antisense strand or portion and is substantially identical in sequence to the targeted portion of the target transcript. In general, considerations for selection of the sequence of the antisense strand of an shRNA molecule are similar to those for selection of the sequence of the antisense strand of an siRNA molecule that targets the same transcript.

In some embodiments, the silencing element comprises or encodes an antisense oligonucleotide. An “antisense oligonucleotide” is a single-stranded nucleic acid sequence that is wholly or partially complementary to a target gene, and can be DNA, or its RNA counterpart (i.e., wherein T residues of the DNA are U residues in the RNA counterpart).

The antisense oligonucleotides of this invention are designed to be hybridizable with target RNA (e.g., mRNA) or DNA. For example, an oligonucleotide (e.g., DNA oligonucleotide) that hybridizes to an mRNA molecule can be used to target the mRNA for RnaseH digestion. Alternatively, an oligonucleotide that hybridizes to the translation initiation site of an mRNA molecule can be used to prevent translation of the mRNA. In another approach, oligonucleotides that bind to double-stranded DNA can be administered. Such oligonucleotides can form a triplex construct and inhibit the transcription of the DNA. Triple helix pairing prevents the double helix from opening sufficiently to allow the binding of polymerases, transcription factors, or regulatory molecules. Such oligonucleotides of the invention can be constructed using the base-pairing rules of triple helix formation and the nucleotide sequences of the target genes.

As non-limiting examples, antisense oligonucleotides can be targeted to hybridize to the following regions: mRNA cap region, translation initiation site, translational termination site, transcription initiation site, transcription termination site, polyadenylation signal, 3′ untranslated region, 5′ untranslated region, 5′ coding region, mid coding region, and 3′ coding region. In some embodiments, the complementary oligonucleotide is designed to hybridize to the most unique 5′ sequence of a gene, including any of about 15-35 nucleotides spanning the 5′ coding sequence.

Accordingly, the antisense oligonucleotides in accordance with this invention can comprise from about 10 to about 100 nucleotides, including, but not limited to about 10, about 11, about 12, about 13, about 14, about 15, about 16, about 17, about 18, about 19, about 20, about 21, about 22, about 23, about 24, about 25, about 30, about 40, about 50, about 60, about 70, about 80, about 90, or about 100 nucleotides.

Antisense nucleic acids can be produced by standard techniques (see, for example, Shewmaker et al., U.S. Pat. No. 5,107,065). Appropriate oligonucleotides can be designed using OLIGO software (Molecular Biology Insights, Inc., Cascade, Colo.; http://www.oligo.net).

In particular embodiments of the methods of the invention, a TEL2 gene is targeted by a silencing element. As used herein, a target gene or target transcript is “targeted” by a silencing element when the introduction or the expression of the silencing element results in the substantially specific reduction or inhibition in the expression of the target gene and target transcript. The specific region of the target gene or target transcript that has substantial sequence identity or similarity or is complementary to the silencing element is the region that has been “targeted” by the silencing element.

3. Expression Cassettes and Transgenic Animals

As discussed above, the silencing elements employed in the methods and compositions of the invention can comprise a DNA template for a dsRNA (e.g., shRNA) or antisense RNA. In such embodiments, the DNA molecule encoding the dsRNA or antisense RNA is found in an expression cassette. In addition, polynucleotides that comprise a coding sequence for a polypeptide (e.g., antibody that inhibits TEL2 activity) can be found in an expression cassette. In certain embodiments, a polynucleotide that encodes a TEL2 polypeptide can be found in an expression cassette.

The expression cassettes can comprise one or more regulatory sequences that are operably linked to the nucleotide sequence encoding the silencing element or polypeptide that facilitate expression of the polynucleotide or polypeptide. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. See, for example, Goeddel (1990) in Gene Expression Technology: Methods in Enzymology 185 (Academic Press, San Diego, Calif.). Regulatory sequences may include promoters, translation leader sequences, introns, and polyadenylation recognition sequences.

Regulatory sequences are operably linked with a coding sequence to allow for expression of the polypeptide encoded by the coding sequence or to allow for the expression of the encoded polynucleotide silencing element. “Operably linked” is intended to mean that the coding sequence (i.e., a DNA encoding a silencing element or a coding sequence for a polypeptide of interest) is functionally linked to the regulatory sequence(s) in a manner that allows for expression of the nucleotide sequence. Operably linked elements may be contiguous or non-contiguous. Polynucleotides may be operably linked to regulatory sequences in sense or antisense orientation.

The regulatory regions (i.e., promoters, transcriptional regulatory regions, and translational termination regions) and/or the coding polynucleotides may be native/analogous to the cell to which the polynucleotide is being introduced or to each other. Alternatively, the regulatory regions and/or the coding polynucleotides may be heterologous to the cell to which the polynucleotide is being introduced or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, a sequence that is heterologous to a cell is a sequence that originates from a foreign species, or, if from the same species, is substantially modified in the cell from its native form in composition and/or genomic locus by deliberate human intervention.

Regulatory sequences include those that direct constitutive expression of a nucleotide sequence in many types of host cells and those that direct expression of the nucleotide sequence only in certain host cells (e.g., tissue-specific regulatory sequences) or at particular stages of development/differentiation (e.g., development-specific regulatory sequences), or those that are chemically-induced. It will be appreciated by those skilled in the art that the design of the expression cassette can depend on such factors as the choice of the host cell to which the polynucleotide is to be introduced, the level of expression of the silencing element or polypeptide desired, and the like. Such expression cassettes typically include one or more appropriately positioned sites for restriction enzymes, to facilitate introduction of the nucleic acid into a vector.

It will further be appreciated that appropriate promoter and/or regulatory elements can readily be selected to allow expression of the relevant transcription units/silencing elements in the cell of interest and at the particular developmental/differentiation state. In certain embodiments, the promoter utilized to direct intracellular expression of a silencing element is a promoter for RNA polymerase III (Pol III). References discussing various Pol III promoters, include, for example, Yu et al. (2002) Proc. Natl. Acad. Sci. 99(9), 6047-6052; Sui et al. (2002) Proc. Natl. Acad. Sci. 99(8), 5515-5520 (2002); Paddison et al. (2002) Genes and Dev. 16, 948-958; Brummelkamp et al. (2002) Science 296, 550-553; Miyagashi (2002) Biotech. 20, 497-500; Paul et al. (2002) Nat. Biotech. 20, 505-508; Tuschl et al. (2002) Nat. Biotech. 20, 446-448. According to other embodiments, a promoter for RNA polymerase I, e.g., a tRNA promoter, can be used for expression of the silencing element. See McCown et al. (2003) Virology 313(2):514-24; Kawasaki (2003) Nucleic Acids Res. 31 (2):700-7. In some embodiments in which the polynucleotide comprises a coding sequence for a polypeptide, a promoter for RNA polymerase II can be used.

The regulatory sequences can also be provided by viral regulatory elements. For example, commonly used promoters are derived from polyoma, Adenovirus 2, cytomegalovirus, and Simian Virus 40. For other suitable expression systems for eukaryotic cells, see Chapters 16 and 17 of Sambrook et al. (1989) Molecular Cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.).

Various constitutive promoters are known. For example, in various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art can be used to achieve expression of a coding sequence of interest. Promoters which may be used include, but are not limited to, the long terminal repeat as described in Squinto et al. (1991) Cell 65:1 20); the SV40 early promoter region (Bernoist and Chambon (1981) Nature 290:304 310), the CMV promoter, the M-MuLV 5′ terminal repeat the promoter contained in the 3′ long terminal repeat of Rous sarcoma virus (Yamamoto et al. (1980) Cell 22:787 797), and the herpes thymidine kinase promoter (Wagner et al. (1981) Proc. Natl. Acad. Sci. U.S.A. 78:144 1445).

Inducible promoters are also known. Non-limiting examples of inducible promoters and their inducer include MT II/Phorbol Ester (TPA) (Palmiter et al. (1982) Nature 300:611) and heavy metals (Haslinger and Karin (1985) Proc. Nat'l Acad. Sci. USA. 82:8572; Searle et al. (1985) Mol. Cell. Biol. 5:1480; Stuart et al. (1985) Nature 317:828; Imagawa et al. (1987) Cell 51:251; Karin et al. (1987) Mol. Cell. Biol. 7:606; Angel et al. (1987) Cell 49:729; McNeall et al. (1989) Gene 76:8); MMTV (mouse mammary tumor virus)/Glucocorticoids (Huang et al. (1981) Cell 27:245; Lee et al. (1981) Nature 294:228; Majors and Varmus (1983) Proc. Nat'l Acad. Sci. USA. 80:5866; Chandler et al. (1983) Cell 33:489; Ponta et al. (1985) Proc. Nat'l Acad. Sci. USA. 82:1020; Sakai et al. (1988) Genes and Dev. 2:1144); β-Interferon/poly(rI)X and poly(rc) (Tavernier et al. (1983) Nature 301:634); Adenovirus 5 E2/E1A (Imperiale and Nevins (1984) Mol. Cell. Biol. 4:875); c-jun/Phorbol Ester (TPA), H₂O₂; Collagenase/Phorbol Ester (TPA) (Angel et al. (1987) Mol. Cell. Biol. 7:2256); Stromelysin/Phorbol Ester (TPA), IL-1 (Angel et al. (1987) Cell 49:729); SV40/Phorbol Ester (TPA) (Angel et al. (1987) Cell 49:729); Murine MX Gene/Interferon, Newcastle Disease Virus; GRP78 Gene/A23187 (Resendez Jr. et al. (1988) Mol. Cell. Biol. 8:4579); α-2-Macroglobulin/IL-6; Vimentin/Serum (Kunz et al. (1989) Nucl. Acids Res. 17:1121); MHC Class I Gene H-2 kB/Interferon (Blanar et al. (1989) EMBO J. 8:1139); HSP70/E1a, SV40 Large T Antigen (Taylor and Kingston (1990) Mol. Cell. Biol. 10:165; Taylor and Kingston (1990) Mol. Cell. Biol. 10:176; Taylor et al. (1989) J. Biol. Chem. 264:15160); Proliferin/Phorbol Ester-TPA (Mordacq and Linzer (1989) Genes and Dev. 3:760); Tumor Necrosis Factor/PMA (Hensel et al. (1989) Lymphokine Res. 8:347); Thyroid Stimulating Hormone α Gene/Thyroid Hormone (Chatterjee et al. (1989) Proc. Nat'l Acad. Sci. USA. 86:9114); and, Insulin E Box/Glucose.

A variety of translation control elements are known to those of ordinary skill in the art and can be used in the presently disclosed methods and compositions. These include, but are not limited to, ribosome binding sites, translation initiation and termination codons, and elements derived from picornaviruses (particularly an internal ribosome entry site, or IRES, also referred to as a CITE sequence).

In general, expression vectors of utility in recombinant DNA techniques are often in the form of plasmids (vectors). However, the invention is intended to include such other forms of expression vectors, such as viral vectors (e.g., replication defective retroviruses, adenoviruses, lentiviruses, and adeno-associated viruses). See, for example, U.S. Publication 2005214851, herein incorporated by reference. Retroviral vectors, particularly lentiviral vectors, are transduced by packaging the vectors into virions prior to contact with a cell.

An expression cassette can further comprise a selection marker. As used herein, the term “selection marker” comprises any polynucleotide, which when expressed in a cell allows for the selection of the transformed cell with the vector. For example, a selection marker can confer resistance to a drug, a nutritional requirement, or a cytotoxic drug. A selection marker can also induce a selectable phenotype such as fluorescence or a color deposit. A “positive selection marker” allows a cell expressing the marker to survive against a selective agent and thus confers a positive selection characteristic onto the cell expressing that marker. Positive selection marker/agents include, for example, Neo/G418, Neo/Kanamycin, Hyg/Hygromycin, hisD/Histidinol, Gpt/Xanthine, Ble/Bleomycin, HPRT/Hypoxanthine. Other positive selection markers include DNA sequences encoding membrane bound polypeptides. Such polypeptides are well known to those skilled in the art and can comprise, for example, a secretory sequence, an extracellular domain, a transmembrane domain and an intracellular domain. When expressed as a positive selection marker, such polypeptides associate with the cell membrane. Fluorescently labeled antibodies specific for the extracellular domain may then be used in a fluorescence activated cell sorter (FACS) to select for cells expressing the membrane bound polypeptide. In some of the embodiments wherein the expression cassette further comprises a selectable marker, an internal ribosome entry site, or IRES, also referred to as a CITE sequence can be used to separate the coding sequences of the selectable marker and the polypolypeptide of interest (e.g., PAX6, CRX), which allows for simultaneous transcription of the two sequences under the control of the same promoter sequences, but separate translation of the transcripts into polypeptides.

A “negative selection marker” allows the cell expressing the marker to not survive against a selective agent and thus confers a negative selection characteristic onto the cell expressing the marker. Negative selection marker/agents include, for example, HSV-tk/Acyclovir or Gancyclovir or FIAU, Hprt/6-thioguanine, Gpt/6-thioxanthine, cytosine deaminase/5-fluoro-cytosine, diphtheria toxin or the ricin toxin. See, for example, U.S. Pat. No. 5,464,764, herein incorporated by reference.

The present invention further provides transgenic animals comprising a heterologous polynucleotide encoding a TEL2 polypeptide or an active variant or fragment thereof. Such animals are useful as animal models for cancer and in particular, are useful in methods for screening compounds to identify those that inhibit tumor incidence or growth, or reduce tumor size. Transgenic rodents that comprise a human TEL2-encoding polynucleotide are especially useful as a model for human cancer because rodents do not have a TEL2 gene. In general, methods of generating transgenic animals are well known in the art (for example, see Grosveld et al., Transgenic Animals, Academic Press Ltd., San Diego, Calif. (1992), which is herein incorporated by reference in its entirety).

In certain embodiments, the transgenic animal comprises a single copy of the polynucleotide encoding the TEL2 polypeptide or an active variant or fragment thereof (i.e., is heterozygous for the TEL2 coding sequence). In particular embodiments, the transgenic animal comprises a polynucleotide that encodes a polypeptide having the sequence set forth in SEQ ID NO: 4 or an active variant or fragment thereof. The polynucleotide can be a human TEL2-encoding genomic sequence. In some of these embodiments, the polynucleotide encoding the TEL2 polypeptide or active variant or fragment thereof further comprises a TEL2 promoter sequence and, in some embodiments, other regulatory sequences operably linked to the TEL2-encoding polynucleotide, so that the expression of TEL2 is under the regulation of its own promoter. In particular embodiments, the TEL2-encoding polynucleotide comprises the upstream genomic sequence of a TEL2 coding sequence. In particular embodiments, the TEL2-encoding polynucleotide comprises about 1 kb, about 2 kb, about 3 kb, about 4 kb, about 5 kb, about 6 kb, about 7 kb, about 8 kb, about 9 kb, about 10 kb, about 11 kb, about 12 kb, about 13 kb, about 14 kb, about 15 kb, or more of upstream genomic sequence from the TEL2 coding sequence. In some embodiments, the TEL2-encoding polynucleotide comprises about 1 kb, about 5 kb, about 10 kb, about 15 kb, about 20 kb, about 25 kb, about 30 kb, about 35 kb, about 40 kb, about 45 kb, about 50 kb, or more of downstream genomic sequence from the TEL2 coding sequence. In certain embodiments, the transgenic animal comprises about 10 kb of upstream genomic sequence, the human TEL2-encoding genomic sequence, and about 30 kb of downstream genomic sequence.

In some embodiments, the transgenic animal further comprises a mutation in at least one copy of the gene that encodes the tumor suppressor p53 that inhibits the activity of p53 (i.e., the transgenic animal is heterozygous for a p53 mutation). The p53 polypeptide functions as a tumor suppressor by activating DNA repair proteins, inducing growth arrest by inhibiting cell cycle progression, and initiating apoptosis.

In some of these embodiments, the transgenic animal is heterozygous for a p53 null mutation (i.e., no active p53 polypeptide is produced from this allele). In some of these embodiments, the mutated p53 produced from the mutant allele does not function in a dominant negative manner. Therefore, these animals comprise one allele having a p53 null mutation that does not produce an active p53 polypeptide and another allele that produces an active p53 polypeptide. A non-limiting example of such a null p53 mutation is the mutation described in Jacks et al. (1994) Current Biology 4:1-7, which is herein incorporated by reference in its entirety, which replaced exons 2 through 7 of the p53 gene with a neomycin resistance gene expression cassette. Other p53 mutations are known in the art (see, for example, Hollstein et al. (1991) Science 253:49-53; Soussi (2007) Cancer Cell 12(4):303-12; Cheung (2009) Br J Haematol 146:257-69; Pfeifer et al. (2009) Hum Genet. 125:493-506; Petitjean et al. (2007) Oncogene 26:2157-65; each of which are herein incorporated by reference in its entirety).

In some embodiments, the transgenic animal is not a human. Non-limiting animals include cattle, sheep, goats, pigs, horses, rabbits, dogs, monkeys, cats, mice, rats, rabbits, and chickens. In particular embodiments, the transgenic animal is a rodent. Non-limiting examples of rodents include mice, rats, hamsters, guinea pigs. In some of these embodiments, the transgenic animal is a mouse.

Such methods of the invention involve introducing a polypeptide or polynucleotide into a cell. “Introducing” is intended to mean presenting to the cell the polynucleotide or polypeptide in such a manner that the sequence gains access to the interior of a cell. The methods of the invention do not depend on a particular method for introducing a sequence into a cell, only that the polynucleotide or polypeptides gains access to the interior of a cell. Methods for introducing polynucleotide or polypeptides into various cell types are known in the art including, but not limited to, stable transformation methods, transient transformation methods, and virus-mediated methods.

The transgenic animals have stably incorporated into its genome the TEL2-encoding polynucleotide. “Stable transformation” is intended to mean that the nucleotide construct introduced into a cell integrates into the DNA of the cell and is capable of being inherited by the progeny thereof “Transient transformation” is intended to mean that a polynucleotide is introduced into the cell and does not integrate into the genome of the cell or a polypeptide is introduced into a cell. Transformation protocols as well as protocols for introducing polypeptides or polynucleotide sequences into cell may vary depending on the type of cell targeted for transformation.

Exemplary art-recognized techniques for introducing foreign polynucleotides into a host cell, include, but are not limited to, calcium phosphate or calcium chloride co-precipitation, DEAE-dextran-mediated transfection, lipofection, particle gun, or electroporation and viral vectors. Suitable methods for transforming or transfecting host cells can be found in U.S. Pat. No. 5,049,386, U.S. Pat. No. 4,946,787; and U.S. Pat. No. 4,897,355, Sambrook et al. (1989) Molecular Cloning: A Laboratory Manual (2d ed., Cold Spring Harbor Laboratory Press, Plainview, N.Y.) and other standard molecular biology laboratory manuals. Various transfection agents can be used in these techniques. Such agent are known, see for example, WO 2005012487. One of skill will recognize that depending on the method by which a polynucleotide is introduced into a cell, the silencing element can be stably incorporated into the genome of the cell, replicated on an autonomous vector or plasmid, or present transiently in the cell. Viral vector delivery systems include DNA and RNA viruses, which have either episomal or integrated genomes after delivery to the cell. For a review of viral vector procedures, see Anderson (1992) Science 256:808-813; Haddada et al. (1995) Current Topics in Microbiology and Immunology Doerfler and Bohm (eds); and Yu et al. (1994) Gene Therapy 1:13-26. Conventional viral based systems for the delivery of polynucleotides could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene.

4. mTORC3 Binding Agents and mTORC3 Modulating Agents

i. mTORC3 Modulating Agents

As used herein, the term “modulating” includes “inducing”, “inhibiting”, “potentiating”, “elevating”, “increasing”, “decreasing” or the like. Each of these terms denote a quantitative difference between two states and in particular, refer to at least a statistically significant difference between the two states.

The term “mTORC3 agonist” refers to an agent which potentiates, induces or otherwise enhances one or more of the biological activities of the mTORC3 complex. The activity increases by a statistically significant amount including, for example, an increase of at least about 5%, 10%, 15%, 20%, 25%, 30%, 35%, 40%, 45%, 50%, 55%, 60%, 65%, 70%, 75%, 80%, 85%, 95% or 100% of the activity of the mTORC3 complex compared to an appropriate control. mTORC3 agonists enhance the proliferation of cells, which find use in various biotechnological applications, such as the transformation or infection of slow-growing cells.

The term “mTORC3 antagonist” refers to an agent that reduces, inhibits, or otherwise diminishes one or more of the biological activities of the mTORC3 complex. Antagonism using the mTORC3 antagonist does not necessarily indicate a total elimination of the mTORC3 activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of the mTORC3 complex compared to an appropriate control. As discussed in more detail elsewhere herein, mTORC3 antagonists find use in reducing cellular growth and survival, especially for the treatment of conditions associated with unregulated cellular growth, such as cancer. Further uses include the treatment and prevention of Epstein-Barr virus infection.

By “specific modulating agent” is intended an agent that modulates the activity of a defined target. Thus, an mTORC3 specific modulating agent modulates the biological activity of mTORC3 by a statically significant amount (i.e., at least 5%, 10%, 20%, 30%, 40%, 50%, 60%, 70%, 80%, 90%, 100% or greater) and the agent does not modulate the biological activity of any monomeric subunits, or non-mTORC3 complexes which comprise either mTOR or TEL2 by a statistically significant amount (i.e., the activity is modulated by less than 5%, 4%, 3%, 2% or 1%). One of skill will be aware of the proper controls that are needed to carry out such a determination. An mTORC3 specific modulating agent may or may not be an mTORC3 specific binding agent. A specific modulating agent may be an agonist or an antagonist.

In some embodiments, the mTORC3 antagonist is one that inhibits the association of mTOR and TEL2 (either direct or indirect association), which can be through binding to mTOR or TEL2 and inhibiting their association with each other or with the mTORC3 complex. As a non-limiting example, the mTORC3 antagonist can bind to the domain of mTOR that is utilized for the association of mTOR with TEL2 or with the mTORC3 complex or through binding to the domain of TEL2 that is utilized for the association of TEL2 with mTOR or with the mTORC3 complex, thus blocking the formation of the association between TEL2 and mTOR or the general formation of the mTORC3 complex. In some of these embodiments, the mTORC3 antagonist binds to the pointed (PNT) domain or the Ets domain of the TEL2 polypeptide. In other embodiments, the mTORC3 antagonist binds to at least one HEAT repeat of mTOR.

Ii. mTORC3 Binding Agents

As used herein, an “mTORC3 binding agent” refers to any compound that directly interacts with or binds to the mTORC3 complex. By “specific binding agent” is intended an agent that binds substantially only to a defined target. Thus, an mTORC3 specific binding agent interacts directly with mTORC3 and, in some embodiments, binds substantially only to epitopes which are formed upon the association of mTOR with TEL2 in the mTORC3 complex. Thus, an mTORC3 specific binding agent will not substantially interact with monomeric protein subunits of the mTORC3 or non-mTORC3 complexes comprising mTOR or TEL2 in a statistically significant amount. By “specifically binds to an mTOR complex 3 (mTORC3)” is intended that the binding agent has a binding affinity for a non-mTORC3 epitope which is less than 10%, 9%, 8%, 7%, 6%, 5%, 4%, 3%, 2% or 1% of the binding affinity for the unique mTORC3 epitope. One of skill will be aware of the proper controls that are needed to carry out such a determination. An mTORC3 specific binding agent may or may not modulate the activity of mTORC3.

By “mTORC3 specific binding/modulating agent” is intended an agent that possesses the properties of both an mTORC3 specific binding agent and an mTORC3 specific modulating agent.

In one embodiment, the mTORC3 binding and/or modulating agent is a small molecule, which can be an organic or inorganic compound (i.e., including heteroorganic and organometallic compounds). The mTORC3 binding and/or modulating agent can also be a peptide, peptidomimetic, amino acid, amino acid analog, polynucleotide, polynucleotide analog, nucleotide, nucleotide analog, or a lipid.

a. Anti-mTORC3 Antibodies

As noted herein, the invention includes antibodies that specifically bind to the mTOR complex 3 (mTORC3). Antibodies, including monoclonal antibodies (mAbs), can be made by standard protocols. See, for example, Harlow and Lane, Using Antibodies: A Laboratory Manual, CSHL, New York, 1999. Briefly, a mammal such as a mouse, hamster or rabbit can be immunized with an immunogenic form of a peptide or a peptide complex. Techniques for conferring immunogenicity on a protein or peptide include conjugation to carriers or other techniques, well known in the art. In particular embodiments, the subject antibodies are immunospecific for the unique antigenic determinants of mTORC3.

As discussed herein, these antibodies are collectively referred to as “anti-mTORC3 antibodies”. Thus, by “anti-mTORC3 antibodies” is intended antibodies specific for mTORC3. All of these antibodies are encompassed by the discussion herein. The respective antibodies can be used alone or in combination in the methods of the invention.

By “antibodies that specifically bind” is intended that the antibodies will not substantially cross react with another polypeptide or polypeptide complex. By “not substantially cross react” is intended that the antibody or fragment has a binding affinity for a different protein complex which is less than 10%, less than 5%, or less than 1%, of the binding affinity for the mTORC3 complex.

In specific embodiments, the anti-mTORC3 antibody binds specifically to mTORC3 and reduces the activity of the mTORC3 complex. Thus, in specific embodiments, the anti-mTORC3 antibody is an mTORC3 antagonist.

The anti-mTORC3 antibodies disclosed herein and for use in the methods of the present invention can be produced using any antibody production method known to those of skill in the art. Thus, polyclonal sera may be prepared by conventional methods. In general, a solution containing the mTORC3 complex or a portion thereof is first used to immunize a suitable animal, preferably a mouse, rat, rabbit, or goat. Rabbits or goats are preferred for the preparation of polyclonal sera due to the volume of serum obtainable, and the availability of labeled anti-rabbit and anti-goat antibodies.

Polyclonal sera can be prepared in a transgenic animal, preferably a mouse bearing human immunoglobulin loci. In a preferred embodiment, Sf9 (Spodoptera frugiperda) cells expressing mTOR and TEL2 and in some embodiments, other members of the mTORC3 complex, are used as the immunogen. Immunization can also be performed by mixing or emulsifying the antigen-containing solution in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antisera are obtained by bleeding the immunized animal into a glass or plastic container, incubating the blood at 25° C. for one hour, followed by incubating at 4° C. for 2-18 hours. The serum is recovered by centrifugation (e.g., 1,000×g for 10 minutes). About 20-50 ml per bleed may be obtained from rabbits.

Production of the Sf9 cells is disclosed in U.S. Pat. No. 6,004,552. Briefly, sequences encoding the mTORC3 complex (e.g., sequences encoding mTOR and TEL2) are recombined into a baculovirus using transfer vectors. The plasmids are co-transfected with wild-type baculovirus DNA into Sf9 cells. Recombinant baculovirus-infected Sf9 cells are identified and clonally purified.

In some embodiments, the antibody is monoclonal in nature. By “monoclonal antibody” is intended an antibody obtained from a population of substantially homogeneous antibodies, that is, the individual antibodies comprising the population are identical except for possible naturally occurring mutations that may be present in minor amounts. The term is not limited regarding the species or source of the antibody. The term encompasses whole immunoglobulins as well as fragments such as Fab, F(ab′)2, Fv, and others which retain the antigen binding function of the antibody. Monoclonal antibodies are highly specific, being directed against a single antigenic site on the target polypeptide. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein (Nature 256:495-97, 1975), or may be made by recombinant DNA methods (see, e.g., U.S. Pat. No. 4,816,567). The “monoclonal antibodies” may also be isolated from phage antibody libraries using the techniques described in, for example, Clackson et al. (Nature 352:624-28, 1991), Marks et al. (J. Mol. Biol. 222:581-97, 1991) and U.S. Pat. No. 5,514,548.

By “epitope” is intended the part of an antigenic molecule to which an antibody is produced and to which the antibody will bind. Epitopes can comprise linear amino acid residues (i.e., residues within the epitope are arranged sequentially one after another in a linear fashion), nonlinear amino acid residues (referred to herein as “nonlinear epitopes”—these epitopes are not arranged sequentially), or both linear and nonlinear amino acid residues. For purposes of the presently disclosed subject matter, the epitope that is recognized by the specific anti-mTORC3 antibodies is one that is formed upon complex formation and is not present in either the TEL2 or mTOR polypeptide alone.

As discussed herein, mAbs can be prepared using the method of Kohler and Milstein, or a modification thereof. Typically, a mouse is immunized with a solution containing an antigen. Immunization can be performed by mixing or emulsifying the antigen-containing solution in saline, preferably in an adjuvant such as Freund's complete adjuvant, and injecting the mixture or emulsion parenterally. Any method of immunization known in the art may be used to obtain the monoclonal antibodies of the invention. After immunization of the animal, the spleen (and optionally, several large lymph nodes) are removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial dilution and are assayed for the production of antibodies that specifically bind the antigen of interest (and that do not bind to unrelated antigens). The selected mAb-secreting hybridomas are then cultured either in vitro (e.g., in tissue culture bottles or hollow fiber reactors), or in vivo (as ascites in mice).

Where the anti-mTORC3 antibodies of the invention are to be prepared using recombinant DNA methods, the DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells described herein can serve as a source of such DNA. Once isolated, the DNA can be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding an antibody includes Skerra (1993) Curr. Opinion in Immunol. 5:256-62; and Phickthun (1992) Immunol. Revs. 130:151-88. Alternatively, antibody can be produced in a cell line such as a CHO cell line, as disclosed in U.S. Pat. Nos. 5,545,403; 5,545,405 and 5,998,144. Briefly the cell line is transfected with vectors capable of expressing a light chain and a heavy chain, respectively. By transfecting the two proteins on separate vectors, chimeric antibodies can be produced. Another advantage is the correct glycosylation of the antibody.

Additionally, the term “anti-mTORC3 antibody” as used herein encompasses chimeric and humanized anti-mTORC3 antibodies. By “chimeric” antibodies is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to the mTORC3 antigen. The non-human source can be any vertebrate source that can be used to generate antibodies to a human mTORC3 antigen or material comprising a human mTORC3 antigen. Such non-human sources include, but are not limited to, rodents (e.g., rabbit, rat, mouse, etc.; see, e.g., U.S. Pat. No. 4,816,567) and non-human primates (e.g., Old World Monkeys, Apes, etc.; see, e.g., U.S. Pat. Nos. 5,750,105 and 5,756,096). As used herein, the phrase “immunologically active” when used in reference to chimeric/humanized anti-mTORC3 antibodies means chimeric/humanized antibodies that bind mTORC3.

By “humanized” is intended forms of anti-mTORC3 antibodies that contain minimal sequence derived from non-human immunoglobulin sequences. For the most part, humanized antibodies are human immunoglobulins (recipient antibody) in which residues from a hypervariable region (also known as complementarity determining region or CDR) of the recipient are replaced by residues from a hypervariable region of a non-human species (donor antibody) such as mouse, rat, rabbit, or nonhuman primate having the desired specificity, affinity, and capacity. The phrase “complementarity determining region” refers to amino acid sequences which together define the binding affinity and specificity of the natural Fv region of a native immunoglobulin binding site. See, for example, Chothia et al. (1987) J. Mol. Biol. 196:901-17; and Kabat et al. (U.S. Dept. of Health and Human Services, NIH Publication No. 91-3242, 1991). The phrase “constant region” refers to the portion of the antibody molecule that confers effector functions.

Humanization can be essentially performed following the methods described by Jones et al. (1986) Nature 321:522-25; Riechmann et al. (1988) Nature 332:323-27; and Verhoeyen et al. (1988) Science 239:1534-36, by substituting rodent or mutant rodent CDRs or CDR sequences for the corresponding sequences of a human antibody. See also U.S. Pat. Nos. 5,225,539; 5,585,089; 5,693,761; 5,693,762; and 5,859,205. In some instances, residues within the framework regions of one or more variable regions of the human immunoglobulin are replaced by corresponding non-human residues (see, for example, U.S. Pat. Nos. 5,585,089; 5,693,761; 5,693,762; and 6,180,370). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. Accordingly, such “humanized” antibodies may include antibodies wherein substantially less than an intact human variable domain has been substituted by the corresponding sequence from a non-human species.

Also encompassed by the term “anti-mTORC3 antibodies” are xenogeneic or modified anti-mTORC3 antibodies produced in a non-human mammalian host, more particularly a transgenic mouse, characterized by inactivated endogenous immunoglobulin loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. Nos. 5,877,397 and 5,939,598. Preferably, fully human antibodies to mTORC3 can be obtained by immunizing transgenic mice. One such mouse is disclosed in U.S. Pat. Nos. 6,075,181; 6,091,001; and 6,114,598.

Fragments of the anti-mTORC3 antibodies are suitable for use in the methods of the invention so long as they retain the desired affinity of the full-length antibody. Thus, a fragment of an anti-mTORC3 antibody will retain the ability to specifically bind to mTORC3. Such fragments are characterized by properties similar to the corresponding full-length anti-mTORC3 antibody; that is, the fragments will specifically bind mTORC3. Such fragments are referred to herein as “antigen-binding” fragments.

Suitable antigen-binding fragments of an antibody comprise a portion of a full-length antibody, generally the antigen-binding or variable region thereof. Examples of antibody fragments include, but are not limited to, Fab, F(ab′)₂, and Fv fragments and single-chain antibody molecules. By “Fab” is intended a monovalent antigen-binding fragment of an immunoglobulin that is composed of the light chain and part of the heavy chain. By F(ab′)₂ is intended a bivalent antigen-binding fragment of an immunoglobulin that contains both light chains and part of both heavy chains. By “single-chain Fv” or “sFv” antibody fragments is intended fragments comprising the V_{H} and V_{L} domains of an antibody, wherein these domains are present in a single polypeptide chain. See, for example, U.S. Pat. Nos. 4,946,778; 5,260,203; 5,455,030; and 5,856,456. Generally, the Fv polypeptide further comprises a polypeptide linker between the V_{H} and V_{L} domains that enables the sFv to form the desired structure for antigen binding. For a review of sFv see Pluckthun (1994) in The Pharmacology of Monoclonal Antibodies, Vol. 113, ed. Rosenburg and Moore (Springer-Verlag, New York), pp. 269-315.

Antibodies or antibody fragments can be isolated from antibody phage libraries generated using the techniques described in, for example, McCafferty et al. (1990) Nature 348:552-54; and U.S. Pat. No. 5,514,548. Clackson et al. (1991) Nature 352:624-28; and Marks et al. (1991) J. Mol. Biol. 222:581-97 describe the isolation of murine and human antibodies, respectively, using phage libraries. Subsequent publications describe the production of high affinity (nM range) human antibodies by chain shuffling (Marks et al. (1992) Bio/Technology 10:779-83), as well as combinatorial infection and in vivo recombination as a strategy for constructing very large phage libraries (Waterhouse et al. (1993) Nucleic. Acids Res. 21:2265-66). Thus, these techniques are viable alternatives to traditional monoclonal antibody hybridoma techniques for isolation of monoclonal antibodies.

Various techniques have been developed for the production of antibody fragments. Traditionally, these fragments were derived via proteolytic digestion of intact antibodies (see, e.g., Morimoto et al. (1992) J. Biochem. Biophys. Methods 24:107-17; and Brennan et al. (1985) Science 229:81-3). However, these fragments can now be produced directly by recombinant host cells. For example, the antibody fragments can be isolated from the antibody phage libraries discussed above. Alternatively, Fab fragments can be directly recovered from E. coli and chemically coupled to form F(ab′)₂ fragments (Carter et al. (1992) Bio/Technology 10:163-67). According to another approach, F(ab′)₂ fragments can be isolated directly from recombinant host cell culture. Other techniques for the production of antibody fragments will be apparent to the skilled practitioner.

In still further embodiments, the antibody is bispecific, wherein a first antigen binding domain specifically interacts with an epitope of mTOR and said second antigen binding domain specifically interacts with an epitope of TEL2.

Further provided is a mixture of a first and a second antibody. The mixture comprises a first antibody having a first chemical moiety, wherein the first antibody specifically binds to mTOR or an active variant or fragment thereof, and a second antibody having a second chemical moiety, wherein the second antibody specifically binds to a second polypeptide comprising TEL2 or an active variant or fragment thereof. The chemical moieties of the first and second specific binding agents are those that allow for the detection of an mTOR complex 3, in which the mTOR polypeptide or biologically active variant or fragment thereof and the TEL2 polypeptide or biologically active variant or fragment thereof associate (directly or indirectly) with one another. As a non-limiting example, the chemical moieties of the specific binding agents can be fluorescent molecules (i.e., fluorophores) with overlapping excitation and emission spectra such as those generally used in fluorescence resonance energy transfer (FRET) technology assays, wherein the excitation of a first fluorescent molecule (donor fluorophore) at a first wavelength of light causes the first fluorescent molecule to emit light at a second wavelength, and wherein the second fluorescent molecule (acceptor fluorophore) is excited by the second wavelength of light if the two fluorescent molecules are in close enough proximity to one another, and subsequently, the second fluorescent molecule emits light at a third wavelength, which can be detected using any method or apparatus known in the art. Non-limiting examples of fluorophores that can be conjugated to antibodies include Cy3, Cy5, Cy5.5, Cy7, Alexa488, Alexa555, FITC, and rhodamine (TRITC). It is to be noted that the selection of the donor fluorophore depends on the excitation and emission spectra of the acceptor fluorophore and vice versa. Frequently used fluorophore pairs for FRET include but are not limited to, Cy3 and Cy5, Alexa488 and Alexa555, Alexa488 and Cy3, and FITC and rhodamine.

### II. Uses, Methods, and Kits

The polynucleotides encoding mTOR and TEL2 and active variants and fragments thereof, the TEL2 specific antagonists, and the mTOR complex 3 (mTORC3)-specific binding agents, agonists, and antagonists disclosed herein can be used in one or more of the following methods: (a) screening assays; (b) detection assays; (c) predictive medicine (e.g., diagnostic assays, prognostic assays, monitoring clinical trials, and pharmacogenomics); and (d) methods of treatment (e.g., therapeutic and prophylactic).

1. Methods to Screen for mTORC3 Binding and/or Modulating Agents

The invention provides a method (also referred to herein as a “screening assay”) for identifying binding and/or modulating agents of mTORC3. As discussed herein, identification of various mTORC3 binding agents are of interest, including mTORC3 specific binding agents and mTORC3 agonists and antagonists.

Screening methods for mTORC3 binding agents or mTORC3 agonists or antagonists involve determining if a test compound can bind, specifically or non-specifically, to an mTORC3 complex and/or determining if the test compound can reduce (antagonist) or enhance (agonist) the activity of the mTORC3 complex.

The test compounds employed in the various screening assays can include any candidate compound including, for example, peptides, peptidomimetics, small molecules, antibodies, or other drugs. Such test compounds can be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

Determining whether a test compound can bind to the mTOR complex 3 (mTORC3) can be accomplished, for example, by coupling the test compound with a radioisotope or enzymatic label such that binding of the test compound to the mTORC3 can be determined by detecting the labeled compound in a complex. For example, test compounds can be labeled with ¹²⁵I, ³⁵S, ¹⁴C, or ³H, H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In one embodiment, an assay is a cell-free assay comprising contacting an mTORC3 with a test compound and determining whether the test compound binds to the mTORC3 complex. Binding of the test compound to the mTORC3 complex can be determined either directly or indirectly. An indirect assay could include assaying for a modulation in mTORC3 activity (e.g., phosphorylation of mTORC3 substrates). In a further embodiment, the test or candidate compound specifically binds to or selectively binds to the mTORC3 complex.

In another embodiment, an assay comprises contacting the mTORC3 complex with a test compound and determining the ability of the test compound to reduce or enhance the activity of the mTORC3 complex or portion thereof. Determining the ability of the test compound to reduce or increase the activity of an mTORC3 complex can be accomplished, for example, by determining the ability of the mTORC3 complex to phosphorylate mTORC3 substrates in the presence of the test compound. Such activities are discussed elsewhere herein.

In some assays, it may be desirable to immobilize either an mTORC3 complex or a portion thereof or the test compound to facilitate automation of the assay. In one embodiment, the mTORC3 complex can be immunoprecipitated from a cellular lysate, wherein the complex is bound to a matrix (e.g., beads). In another embodiment, a fusion protein can be provided that adds a domain to the test agent or a subunit of the mTORC3 complex that allows the test agent or the mTORC3 complex to be bound to a matrix. For example, mTORC3 complexes comprising a glutathione-S-transferase/TEL2 fusion protein or a glutathione-S-transferase/mTOR fusion protein can be adsorbed onto glutathione sepharose beads (Sigma Chemical, St. Louis, Mo.) or glutathione-derivatized microtitre plates, which are then combined with the test compound, and the mixture incubated under conditions conducive to complex formation between the test compound and the mTORC3 complex (e.g., at physiological conditions for salt and pH). Following incubation, the beads or microtitre plate wells are washed to remove any unbound components and complex formation of the test compound and mTORC3 complex is measured either directly or indirectly, for example, as described above.

Other techniques for immobilizing proteins on matrices can also be used in the screening assays of the invention. For example, either the mTORC3 complex or the test compound can be immobilized utilizing conjugation of biotin and streptavidin. Biotinylated mTORC3 complexes or test agents can be prepared from biotin-NHS (N-hydroxy-succinimide) using techniques well known in the art (e.g., biotinylation kit, Pierce Chemicals, Rockford, Ill.), and immobilized in the wells of streptavidin-coated 96-well plates (Pierce Chemicals).

In yet another aspect of the invention, the mTOR and/or TEL2 polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with the mTORC3 complex or portions thereof and, in some embodiments, modulate mTORC3 complex activity.

mTORC3 antagonists can also be identified by screening for compounds that specifically inhibit the formation of the mTORC3 complex, such as compounds that bind to TEL2 and prevent TEL2 from associating with mTOR in the mTORC3 complex.

This invention further pertains to novel agents identified by the above-described screening assays and uses thereof as described herein.

2. Methods for Detecting

Various methods and compositions for detecting and/or determining the level of expression of a polynucleotide encoding mTOR or active variants or fragments thereof and a polynucleotide encoding TEL2 or active variants or fragments thereof, or for detecting and/or determining the level of the mTORC3 complex in a sample (e.g., biological sample) are provided. A biological sample can comprise any sample in which one desires to determine the level of expression of a polynucleotide encoding mTOR and a polynucleotide encoding TEL2 or one in which one desires to detect or quantify the level of the mTOR complex 3 (mTORC3).

The term “biological sample” is intended to include tissues, cells, and biological fluids isolated from a subject, as well as tissues, cells, and fluids present within a subject or lysates thereof. That is, the detection method of the invention can be used to detect mTOR mRNA or genomic DNA, TEL2 mRNA or genomic DNA, or the mTORC3 in a biological sample in vitro, as well as, in vivo. For example, in vitro techniques for detection of the mTOR and TEL2 mRNA include, but are not limited to, Northern hybridizations and in situ hybridizations. In vitro techniques for detection of the mTORC3 complex include, but are not limited to, enzyme linked immunosorbent assays (ELISAs), Western blots, immunoprecipitations, and immunofluorescence. In vitro techniques for detection of genomic DNA include Southern hybridizations. Furthermore, in vivo techniques for detection of the mTORC3 complex include, but are not limited to, introducing into a subject a labeled mTORC3 specific binding agent capable of entering the intracellular space of cells. For example, the specific binding agent can be labeled with a radioactive marker whose presence and location in a subject can be detected by standard imaging techniques.

i. Detecting Polynucleotides

In one embodiment, a method for detecting the level of expression of a polynucleotide encoding an mTOR polypeptide or active variants and fragments thereof and a polynucleotide encoding a TEL2 polypeptide or active variants and fragments thereof in a sample comprises contacting the sample with a) a first and a second primer capable of specifically amplifying a first amplicon of a polynucleotide encoding an mTOR polypeptide or an active variant or fragment thereof; and, b) a third and a fourth primer capable of specifically amplifying second amplicon of a polynucleotide encoding a TEL2 polypeptide or an active variant or fragment thereof; wherein the encoded polypeptides are capable of associating with one another in an mTOR complex 3 (mTORC3). The first and the second amplicon is amplified and then detected. In certain embodiments, the first and the second amplicons are of a sufficient length to specifically detect the level of expression of the polynucleotide encoding the mTOR polypeptide or an active variant or fragment thereof and the polynucleotide encoding the TEL2 polypeptide or an active variant or fragment thereof.

In other embodiments, a method for detecting the level of expression of a polynucleotide encoding an mTOR polypeptide or active variants and fragments thereof and a TEL2 polypeptide or active variants and fragments thereof in a sample comprises contacting the sample with a) a first polynucleotide capable of specifically detecting a polynucleotide encoding an mTOR polypeptide or an active variant or fragment thereof; and, b) a second polynucleotide capable of specifically detecting a polynucleotide encoding a TEL2 polypeptide or an active variant or fragment thereof wherein the encoded polypeptides are capable of associating with one another in an mTORC3; and detecting the polynucleotide encoding the mTOR polypeptide or an active variant or fragment thereof and detecting the polynucleotide encoding the TEL2 polypeptide or an active variant or fragment thereof.

In specific embodiments, the sample is contacted with a polynucleotide probe that hybridizes under stringent hybridization conditions to the target sequences to be detected. The sample and probes are then subjected to stringent hybridization conditions and the hybridization of the probe to the target sequences is detected.

Primers and probes are based on the sequence of the polynucleotides encoding mTOR and TEL2 or active variants and fragments thereof. Any conventional nucleic acid hybridization or amplification method can be used to identify the presence of the polynucleotides encoding mTOR and TEL2 in a sample. By “specifically detect” is intended that the polynucleotide can be used as a probe that hybridizes under stringent conditions to a polynucleotide encoding mTOR or TEL2. By “specifically amplify” is intended that the polynucleotide(s) can be used as a primer to specifically amplify an amplicon of a polynucleotide encoding mTOR or TEL2. The level or degree of hybridization which allows for the specific detection of a polynucleotide encoding mTOR or TEL2 is sufficient to distinguish the polynucleotide encoding mTOR or TEL2 from a polynucleotide that does not encode the recited polypeptide. By “shares sufficient sequence identity or complementarity to allow for the amplification of a polynucleotide encoding mTOR or TEL2” is intended the sequence shares at least 80%, 85%, 90%, 91%, 92%, 93%, 94%, 95%, 96%, 97%, 98%, 99% or 100% identity or complementarity to a fragment or across the full length of the polynucleotide encoding mTOR or TEL2.

Regarding the amplification of a target polynucleotide (e.g., by PCR) using a particular amplification primer pair, “stringent conditions” are conditions that permit the primer pair to hybridize to the target polynucleotide to which a primer having the corresponding wild-type sequence (or its complement) would bind and in some embodiments, produce an identifiable amplification product (the amplicon) in a DNA thermal amplification reaction. In a PCR approach, oligonucleotide primers can be designed for use in PCR reactions to amplify a polynucleotide encoding mTOR or TEL2. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Methods of amplification are further described in U.S. Pat. Nos. 4,683,195, 4,683,202 and Chen et al. (1994) PNAS 91:5695-5699. These methods as well as other methods known in the art of DNA amplification may be used in the practice of the embodiments of the present invention. It is understood that a number of parameters in a specific PCR protocol may need to be adjusted to specific laboratory conditions and may be slightly modified and yet allow for the collection of similar results. These adjustments will be apparent to a person skilled in the art. Thermal cyclers are often employed for the specific amplification of polynucleotides. The cycles of denaturation, annealing and polymerization for PCR may be performed using an automated device, typically known as a thermal cycler. Thermal cyclers that may be employed are described elsewhere herein as well as in U.S. Pat. Nos. 5,612,473; 5,602,756; 5,538,871; and 5,475,610, the disclosures of which are herein incorporated by reference.

The amplified polynucleotide (amplicon) can be of any length that allows for the detection of the polynucleotide encoding mTOR or TEL2. For example, the amplicon can be about 10, 50, 100, 200, 300, 500, 700, 100, 2000, 3000, 4000, 5000 nucleotides in length or longer. Further, in some embodiments, the length or sequence of the amplified region (amplicon) of the polynucleotide encoding mTOR or TEL2 is sufficient to distinguish the polynucleotide encoding mTOR or TEL2 from a polynucleotide that does not encode the recited polypeptide.

Any primer or set of primers can be employed in the methods of the invention that allows a polynucleotide encoding an mTOR polypeptide or a TEL2 polypeptide to be amplified and/or detected. For example, in specific embodiments, the first primer pair comprises a first primer comprising a first fragment of a polynucleotide encoding an mTOR polypeptide and a second primer comprising the complement of a second fragment of the polynucleotide encoding the mTOR polypeptide, wherein the first primer pair shares sufficient sequence identity or complementarity to the polynucleotide to specifically amplify the polynucleotide encoding mTOR; and, the second primer pair comprises a first primer comprising a first fragment of a polynucleotide encoding a TEL2 polypeptide and a second primer comprising the complement of a second fragment of the polynucleotide encoding the TEL2 polypeptide, wherein the second primer pair shares sufficient sequence identity or complementarity to the polynucleotide to specifically amplify the polynucleotide encoding TEL2. In specific embodiments, the primer can comprise at least 8, 10, 15, 20, 25, 30, 40 or greater consecutive nucleotides of SEQ ID NO: 1 or 3 or the complement thereof. In order for a nucleic acid molecule to serve as a primer or probe it need only be sufficiently complementary in sequence to be able to form a stable double-stranded structure under the particular solvent and salt concentrations employed.

In hybridization techniques, all or part of a polynucleotide that selectively hybridizes to a target polynucleotide encoding an mTOR polypeptide or a TEL2 polypeptide is employed. By “stringent conditions” or “stringent hybridization conditions” when referring to a polynucleotide probe is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing). Generally, a probe is less than about 1000 nucleotides in length or less than 500 nucleotides in length.

As used herein, a substantially identical or complementary sequence is a polynucleotide that will specifically hybridize to the complement of the nucleic acid molecule to which it is being compared under high stringency conditions. Appropriate stringency conditions which promote DNA hybridization, for example, 6× sodium chloride/sodium citrate (SSC) at about 45° C., followed by a wash of 2×SSC at 50° C., are known to those skilled in the art or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In hybridization reactions, specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.) and Haymes et al. (1985) In: Nucleic Acid Hybridization, a Practical Approach, IRL Press, Washington, D.C.

As used herein, molecules are said to exhibit “complete complementarity” when every nucleotide of one of the polynucleotide molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions.

Ii. Detecting the mTOR Complex 3 (mTORC3)

One aspect of the present invention relates to assays for detecting mTOR complex 3 (mTORC3) in the context of a biological sample. An exemplary method for detecting the presence or absence or the quantity of the mTORC3 in a biological sample involves obtaining a biological sample and contacting the biological sample with a compound or an agent capable of specifically binding and detecting an mTORC3, such that the presence of the mTORC3 is detected in the biological sample. Results obtained with a biological sample from a test subject may be compared to results obtained with a biological sample from a control subject.

As mTORC3 stimulates proliferation, the presence of mTORC3 can be used to detect, separate, or purify proliferating cells.

In one embodiment, an agent for detecting the mTORC3 is an antibody capable of specifically binding to the mTORC3, preferably an antibody with a detectable label. Antibodies can be polyclonal, or more preferably, monoclonal. An intact antibody, or a fragment thereof (e.g., Fab or F(abN)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody.

In some embodiments, the mTORC3 is detected or quantified in a biological sample through the use of a pair of specific binding agents, each of which comprise a chemical moiety, wherein a first specific binding agent specifically binds to an mTOR polypeptide or a biologically active variant or fragment thereof and a second specific binding agent specifically binds to a TEL2 polypeptide or a biologically active variant or fragment thereof. The chemical moieties of the first and second specific binding agents are those that allow for the detection of an mTOR complex 3, in which the mTOR polypeptide or biologically active variant or fragment thereof and the TEL2 polypeptide or biologically active variant or fragment thereof associate (directly or indirectly) with one another. As a non-limiting example, the chemical moieties of the specific binding agents can be fluororescent molecules (i.e., fluorophores) with overlapping excitation and emission spectra, wherein the excitation of a first fluorescent molecule (donor fluorophore) at a first wavelength of light causes the first fluororescent molecule to emit light at a second wavelength, and wherein the second fluorescent molecule (acceptor fluorophore) is excited by the second wavelength of light if the two fluorescent molecules are in close enough proximity to one another, and subsequently, the second fluorescent molecule emits light at a third wavelength, which can be detected using any method or apparatus known in the art. In these embodiments, the method of detecting the mTORC3 complex can further comprise a step of detecting the proximity of the first and second fluorescent molecules through the excitation of the donor fluorophore (e.g., via exposure to a light source) and detection of the emitted light from the acceptor fluorophore using, for example, a fluorescent plate reader. Non-limiting examples of fluorophores and donor/acceptor fluorophore pairs are described elsewhere herein.

3. Kits

As used herein, “kit” refers to a set of reagents for the identification, the detection, and/or the quantification of the polynucleotide encoding an mTOR polypeptide and the polynucleotide encoding a TEL2 polypeptide or detection and/or quantification of the mTOR complex 3 (mTORC3) in biological samples. The terms “kit” and “system,” as used herein are intended to refer to at least one or more detection reagents which, in specific embodiments, are in combination with one or more other types of elements or components (e.g., other types of biochemical reagents, containers, packages, such as packaging intended for commercial sale, substrates to which detection reagents are attached, electronic hardware components, instructions of use, and the like).

In one embodiment, a kit for determining the level of expression of a polynucleotide encoding an mTOR polypeptide and a polynucleotide encoding a TEL2 polypeptide in a sample is provided. The kit comprises a) a first polynucleotide or pair of polynucleotides capable of specifically detecting or amplifying a polynucleotide encoding a first polypeptide encoding an mTOR polypeptide or a biologically active variant or fragment thereof; and, b) a second polynucleotide or pair of polynucleotides capable of specifically detecting or amplifying a polynucleotide encoding a TEL2 polypeptide or a biologically active variant or fragment thereof, wherein the encoded polypeptides are capable of associating with one another in an mTOR complex 3 (mTORC3).

In specific embodiments, the kit comprises a) a first and a second primer that share sufficient sequence homology or complementarity to the polynucleotide encoding an mTOR polypeptide or the active variant or fragment thereof to specifically amplify the polynucleotide encoding an mTOR polypeptide; and, b) a third and a forth primer that share sufficient sequence homology or complementarity to a polynucleotide encoding a TEL2 polypeptide or an active variant or fragment thereof to specifically amplify the polynucleotide encoding a TEL2 polypeptide.

In still other embodiments, the kit comprises a) a first probe that can specifically detect the polynucleotide encoding an mTOR polypeptide or the active variant or fragment thereof, wherein the first probe comprises at least one polynucleotide of a sufficient length of contiguous nucleotides identical or complementary to the polynucleotide encoding an mTOR polypeptide or the active variant thereof; and, b) a second probe that can specifically detect a second polynucleotide encoding a TEL2 polypeptide or an active variant or fragment thereof, wherein the second probe comprises at least one polynucleotide of a sufficient length of contiguous nucleotides identical or complementary to a polynucleotide encoding a TEL2 polypeptide or an active variant or fragment thereof. In still further embodiments, the first polynucleotide hybridizes under stringent conditions to the sequence encoding an mTOR polypeptide or active variant or fragment thereof; and, the second polynucleotide hybridizes under stringent conditions to the sequence encoding a TEL2 polypeptide or an active variant or fragment thereof.

In still other embodiments, a kit for determining the presence of the mTOR complex 3 (mTORC3) in a sample is provided. Such a kit can comprise any mTORC3 specific binding and/or mTORC3 specific binding agent/antagonist disclosed herein, including, but not limited to the mTORC3-specific antibodies disclosed herein or any mixture thereof. In some of these embodiments, the kit further comprises a means for detecting the complex formed between the mTORC3 specific binding agent and mTORC3. As a non-limiting example, in those embodiments wherein the mTORC3 specific binding agent comprises an antibody that specifically binds to mTORC3, the antibody can comprise a detectable label or the kit can comprise a secondary antibody conjugated to a detectable label, wherein the secondary antibody is capable of binding to the mTORC3 antibody.

4. Methods for Diagnosing

Methods for diagnosing the presence of a cancer or determining the severity of a cancer are provided. Such methods can comprise evaluating the level of an mTOR complex 3 (mTORC3) in a biological sample from a subject, comparing the level of mTORC3 in the biological sample from the test subject with the mTORC3 level in an appropriate control, and diagnosing the cancer in the test subject in those instances wherein the mTORC3 level in the biological sample from the test subject is relatively higher than the control, or determining that the cancer of the test subject is more severe than the control in those instances wherein the level of mTORC3 in the test subject is relatively higher than the control.

The term “cancer” refers to the condition in a subject that is characterized by unregulated cell growth, wherein the cancerous cells are capable of local invasion and/or metastasis to noncontiguous sites. As used herein, “cancer cells,” “cancerous cells,” or “tumor cells” refer to the cells that are characterized by this unregulated cell growth and invasive property. The term “cancer” encompasses all types of cancers, including, but not limited to, all forms of carcinomas, melanomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer, acute lymphocytic leukemia (e.g., B-cell acute lymphocytic leukemia), acute myeloid leukemia, ependymoma, Ewing's sarcoma, glioblastoma, medulloblastoma, neuroblastoma, osteosarcoma, rhabdomyosarcoma, rhabdoid cancer, and nephroblastoma (Wilm's tumor).

In some embodiments, the cancer that is being detected is a solid tumor cancer, which refers to cancers that are characterized by a localized mass of tissue that is capable of locally invaded its surrounding tissues or metastasizing to a noncontiguous site. Solid tumor cancers are distinct from leukemias, which are cancers of the blood cells that typically do not form solid masses of cells.

In other embodiments, the cancer is a pediatric cancer, which is a cancer the onset or diagnosis of which occurs during the early stages of life prior to full physical maturity (i.e., embryonic, fetal, infancy, pre-pubertal, adolescent). In some embodiments, the pediatric cancer comprises a pediatric solid tumor cancer. In particular embodiments, the pediatric cancer comprises a pediatric acute lymphocytic leukemia (e.g., B-cell acute lymphocytic leukemia), acute myeloid leukemia, ependymoma, Ewing's sarcoma, glioblastoma, medulloblastoma, neuroblastoma, osteosarcoma, rhabdomyosarcoma, rhabdoid cancer, or nephroblastoma.

In certain embodiments, the cancer that is being detected is a B cell cancer, which is a cancer that is derived from a B cell or B cell precursor, such as a B-cell acute lymphocytic leukemia (B-ALL).

In other embodiments, the diagnostic methods comprise diagnosing or determining the severity of a non-B cell cancer. As used herein, a “non-B cell cancer” is a cancer, such as leukemia or a solid tumor cancer wherein the cancer is not derived from a B cell or B cell precursor. Such methods can comprise the steps of evaluating the expression of TEL2 in a sample from a subject, comparing the expression of TEL2 in the sample from the test subject with the TEL2 expression level in an appropriate control, and diagnosing the cancer in the test subject in those instances wherein the TEL2 expression level in the sample from the test subject is relatively higher than the control, or determining that the cancer of the test subject is more severe than the control in those instances wherein the expression level of TEL2 in the test subject is relatively higher than the control. Determining the TEL2 expression level can comprise measuring the level of TEL2 transcripts or polypeptides in a given biological sample from a test subject or control subject.

A “test subject” is a subject as defined elsewhere herein that has or is suspected of having, or is at risk for developing a cancer or a particular type of cancer. In some instances, the control can be a biological sample obtained from one or more subjects not having or not suspected of having cancer or a particular type of cancer or the control can be a previously assayed value for the same subject (i.e., the test subject and the control subject are the same subject).

In some embodiments, the biological sample is isolated from an organ or tissue that is believed to comprise cancerous cells. In particular embodiments, lysates of isolated cells/tissues, or fluids are prepared and the level of an mTORC3 complex or the expression level of TEL2 is determined within the lysate.

In some embodiments, the steps of the method for detecting or determining the severity of a cancer comprise a step of providing the biological sample and detecting the level of the mTORC3 complex or expression level of TEL2 using the detection methods described elsewhere herein.

The methods described above for evaluating an association between expression level of TEL2 or level of an mTORC3 complex and the presence/severity of a cancer may be performed, wholly or in part, with the use of a computer program or computer-implemented method.

Computer programs and computer program products of the present invention comprise a computer usable medium having control logic stored therein for causing a computer to execute the algorithms disclosed herein. Computer systems of the present invention comprise a processor, operative to determine, accept, check, and display data, a memory for storing data coupled to said processor, a display device coupled to said processor for displaying data, an input device coupled to said processor for entering external data; and a computer-readable script with at least two modes of operation executable by said processor. A computer-readable script may be a computer program or control logic of a computer program product.

It is not critical to the invention that the computer program is written in any particular computer language or to operate on any particular type of computer system or operating system. The computer program may be written, for example, in C++, Java, Perl, Python, Ruby, Pascal, or Basic programming language. It is understood that one may create such a program in one of many different programming languages. In one aspect of this invention, this program is written to operate on a computer utilizing a Linux operating system. In another aspect of this invention, the program is written to operate on a computer utilizing a MS Windows or MacOS operating system.

Those subjects in which cancer has been diagnosed or those subjects that have been determined to have a severe form of cancer can be administered a specific mTORC3 antagonist or a TEL2 antagonist, as described immediately herein below.

5. Methods for Modulating the Activity of the mTORC3 Complex or TEL2

Methods for modulating the activity of the mTORC3 complex or modulating cell growth and/or survival are provided. Such methods can comprise contacting a cell comprising an mTORC3 complex with an mTORC3 agonist or antagonist.

Further, as mTORC3 has been identified in B cells, the contacting of B cells with an mTORC3 antagonist inhibits the growth of the B cell and therefore, can reduce antibody production by activated B cells.

As used herein, “cell growth” refers to cell proliferation, cell division, or progression through the cell cycle. “Cell survival” refers to the ability of a cell to avoid cell death, including both apoptosis and necrosis.

An mTORC3 antagonist will act to reduce cell growth and/or survival, whereas an agonist would enhance cell growth and/or survival. The agonist or antagonist can be an mTORC3 specific binding/modulating agent or an mTORC3 specific modulating agent.

Any method known in the art can be used to measure the growth rate of a cell or an effect on cell survival, including, but not limited to, optical density (OD₆₀₀), CO₂ production, O₂ consumption, assays that measure mitochondrial function, such as those utilizing tetrazolium salts (e.g., MTT, XTT), or other colorimetric reagents (e.g., the WST-1 reagent available from Roche), assays that measure or estimate DNA content, including, but not limited to fluoremetric assays such as those utilizing the fluorescent dye Hoechst 33258, assays that measure or estimate protein content, including, but not limited to, the sulforhodamine B (SRB) assay, manual or automated cell counts (with or without the Trypan Blue stain to distinguish live cells), and clonogenic assays with manual or automated colony counts. Non-limiting examples of assays that can be used to measure levels of apoptosis include, but are not limited to, measurement of DNA fragmentation, caspase activation assays, TUNEL staining, annexin V staining. In some embodiments, the growth rate of a cell is inhibited by an mTORC3 antagonist by at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or higher.

mTORC3 agonists find use in methods in which an enhancement of cellular proliferation is desired, such as the transformation or infection of slow-growing cells.

mTORC3 antagonists find use in treating any unwanted conditions or diseases in which unregulated cellular growth or survival contributes to the condition. For example, the mTORC3 antagonists find use in treating cancers. Thus, in one embodiment, a method of treating a cancer in a subject in need thereof is provided. Such a method comprises administering to a subject in need thereof an effective amount of a specific mTORC3 antagonist. Various mTORC3 antagonists and methods for preparing and identifying such agents are discussed elsewhere herein. In specific embodiments, the antagonist is an antibody or a small molecule.

In some embodiments, the cancer that is being treated with an mTORC3 antagonist is a solid tumor cancer. In other embodiments, the cancer is a pediatric cancer. In some embodiments, the pediatric cancer comprises a pediatric solid tumor cancer. In particular embodiments, the pediatric cancer comprises a pediatric acute lymphocytic leukemia (e.g., B-cell acute lymphocytic leukemia), acute myeloid leukemia, ependymoma, Ewing's sarcoma, glioblastoma, medulloblastoma, neuroblastoma, osteosarcoma, rhabdomyosarcoma, rhabdoid cancer, or nephroblastoma. In other embodiments, the cancer comprises acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, nephroblastoma (Wilm's tumor), hepatocellular carcinoma, esophageal carcinoma, liposarcoma, bladder cancer, gastric cancer, myxofibrosarcoma, colon cancer, kidney cancer, histiosarcoma, ovarian cancer, endometrial carcinoma, lung cancer, or breast cancer.

In particular embodiments, the cancer that can be treated with an mTORC3 antagonist is a B cell cancer. In alternative embodiments, the cancer that is treated with an mTORC3 antagonist is a non-B cell cancer.

The presently disclosed subject matter also provides for methods of treating a non-B cell cancer in a subject in need thereof through the administration of an effective amount of a specific TEL2 antagonist. As discussed elsewhere herein, a TEL2 antagonist can be an antagonist that reduces the expression or activity of TEL2. In some embodiments, the non-B cell cancer that is treated with a specific TEL2 antagonist is ependymoma, Ewing's sarcoma, glioblastoma, medulloblastoma, neuroblastoma, osteosarcoma, rhabdomyosarcoma, rhabdoid cancer, nephroblastoma (Wilm's tumor), esophageal carcinoma, liposarcoma, bladder cancer, gastric cancer, myxofibrosarcoma, colon cancer, kidney cancer, histiosarcoma, ovarian cancer, endometrial carcinoma, lung cancer, or breast cancer.

As TEL2 expression is upregulated in B cells upon Epstein-Barr virus (EBV; also called human herpes virus-4; HHV-4) infection, an mTORC3 antagonist can be administered to subjects to treat or prevent EBV infection. While not being limited by any theory or mechanism of action, it is believed that TEL2, functioning through the mTORC3 complex, may affect the growth of the cell (e.g., B cell) infected by the virus and antagonism of the complex reduces the growth of the cell and, therefore, minimizes growth of the virus. Therefore, an mTORC3 antagonist can be administered to a subject that has been infected with EBV or to a subject at risk for infection by EBV.

A therapeutically effective amount of an mTORC3 or TEL2 antagonist can be administered to a subject. By “therapeutically effective amount” is intended an amount that is useful in the treatment, prevention or diagnosis of a disease or condition. As used herein, a therapeutically effective amount of an mTORC3 antagonist or a TEL2 antagonist is an amount which, when administered to a subject, is sufficient to achieve a desired effect, such as inhibiting cell growth or survival in a subject being treated with that composition. The effective amount of an mTORC3 or TEL2 antagonist useful for inhibiting cell growth or survival will depend on the subject being treated, the severity of the affliction, and the manner of administration of the mTORC3 or TEL2 antagonist.

By “subject” is intended mammals, e.g., primates, humans, agricultural and domesticated animals such as, but not limited to, dogs, cats, cattle, horses, pigs, sheep, and the like. In some embodiments, the subject undergoing treatment with the pharmaceutical formulations of the invention is a human.

When administration is for the purpose of treatment, administration may be for either a prophylactic (i.e., preventative) or therapeutic purpose. When provided prophylactically, the substance is provided in advance of any symptom. The prophylactic administration of the substance serves to prevent or attenuate any subsequent symptom. When provided therapeutically, the substance is provided at (or shortly after) the onset of a symptom. The therapeutic administration of the substance serves to attenuate any actual symptom.

It will be understood by one of skill in the art that the treatment modalities described herein may be used alone or in conjunction with other therapeutic modalities (i.e., as adjuvant therapy), including, but not limited to, surgical therapy, radiotherapy, chemotherapy (e.g., with any chemotherapeutic agent well known in the art) or immunotherapy.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an mTORC3 or TEL2 antagonist can include a single treatment or, preferably, can include a series of treatments. It will also be appreciated that the effective dosage of an mTORC3 or TEL2 antagonist used for treatment may increase or decrease over the course of a particular treatment. Changes in dosage may result and become apparent from the results of diagnostic assays as described herein.

It is understood that appropriate doses of such active compounds depends upon a number of factors within the knowledge of the ordinarily skilled physician, veterinarian, or researcher. The dose(s) of the active compounds will vary, for example, depending upon the identity, size, and condition of the subject or sample being treated, further depending upon the route by which the composition is to be administered, if applicable, and the effect which the practitioner desires the active compound to have upon TEL2 or the mTORC3. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of an active agent depend upon the potency of the active agent with respect to the expression or activity to be modulated. Such appropriate doses may be determined using the assays described herein. When one or more of these small molecules is to be administered to an animal (e.g., a human) in order to reduce the activity of the mTORC3 complex or reduce the expression or activity of TEL2, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Therapeutically effective amounts of an mTORC3 antagonist can be determined by animal studies. When animal assays are used, a dosage is administered to provide a target tissue concentration similar to that which has been shown to be effective in the animal assays. It is recognized that the method of treatment may comprise a single administration of a therapeutically effective amount or multiple administrations of a therapeutically effective amount of the mTORC3 antagonist.

Any delivery system or treatment regimen that effectively achieves the desired effect of inhibiting cell growth can be used. Thus, for example, formulations comprising an effective amount of a pharmaceutical composition of the invention comprising mTORC3 or TEL2 antagonists or anti-mTORC specific binding agents can be used for the purpose of treatment, prevention, and diagnosis of a number of clinical indications related to the activity of the mTORC3 complex.

6. Pharmaceutical Compositions

The mTORC3 specific binding agents, mTORC3 antagonists, and TEL2 antagonists (also referred to herein as “active compounds”) disclosed herein can be incorporated into pharmaceutical compositions suitable for administration. Such compositions typically comprise the compound (e.g., antibody, small molecule) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. In addition, it may be desirable to administer a therapeutically effective amount of the pharmaceutical composition locally to an area in need of treatment (e.g., to an area of the body where inhibiting a T_{R} cell function is desired). This can be achieved by, for example, local or regional infusion or perfusion during surgery, topical application, injection, catheter, suppository, or implant (for example, implants formed from porous, non-porous, or gelatinous materials, including membranes, such as sialastic membranes or fibers), and the like. In one embodiment, administration can be by direct injection at the site (or former site) of a cancer that is to be treated. In another embodiment, the therapeutically effective amount of the pharmaceutical composition is delivered in a vesicle, such as liposomes (see, e.g., Langer (1990) Science 249:1527-33; and Treat et al., in Liposomes in the Therapy of Infectious Disease and Cancer, Lopez Berestein and Fidler (eds.), Liss, N.Y., pp. 353-65, 1989).

In yet another embodiment, the therapeutically effective amount of the pharmaceutical composition can be delivered in a controlled release system. In one example, a pump can be used (see, e.g., Langer (1990) Science 249:1527-33; Sefton (1987) Crit. Rev. Biomed. Eng. 14:201-40; Buchwald et al. (1980) Surgery 88:507-16; Saudek et al. (1989) N. Engl. J. Med. 321:574-79). In another example, polymeric materials can be used (see, e.g., Levy et al. (1985) Science 228:190-92; During et al. (1989) Ann. Neurol. 25:351-56; Howard et al. (1989) J. Neurosurg. 71:105-12). Other controlled release systems, such as those discussed by Langer (1990) Science 249:1527-33, can also be used.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor® EL (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from a pressurized container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### III. Sequence Identity

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

It is to be noted that the term “a” or “an” entity refers to one or more of that entity; for example, “a polypeptide” is understood to represent one or more polypeptides. As such, the terms “a” (or “an”), “one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the presently disclosed subject matter be limited to the specific values recited when defining a range.

The following examples are offered by way of illustration and not by way of limitation.

### EXPERIMENTAL

### Example 1

### Identification and Characterization of the mTOR Complex 3 (mTORC3)

These data show the existence of a novel mTOR-containing protein complex (mTORC), mTORC3, in addition to the previously described mTORC1 and mTORC2. Induction of mTORC3 in cells results in dramatically elevated proliferation and reduced apoptosis. The driver of this complex is the ETS transcription factor TEL2/ETV7, which is a member of mTORC3.

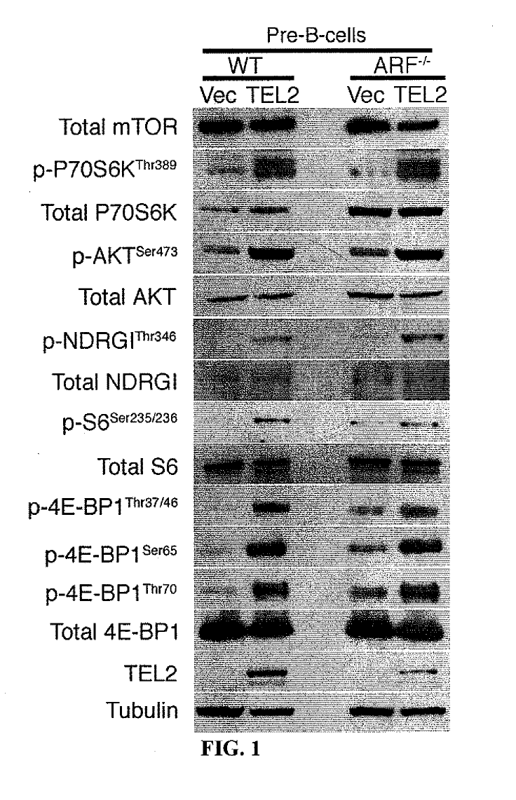

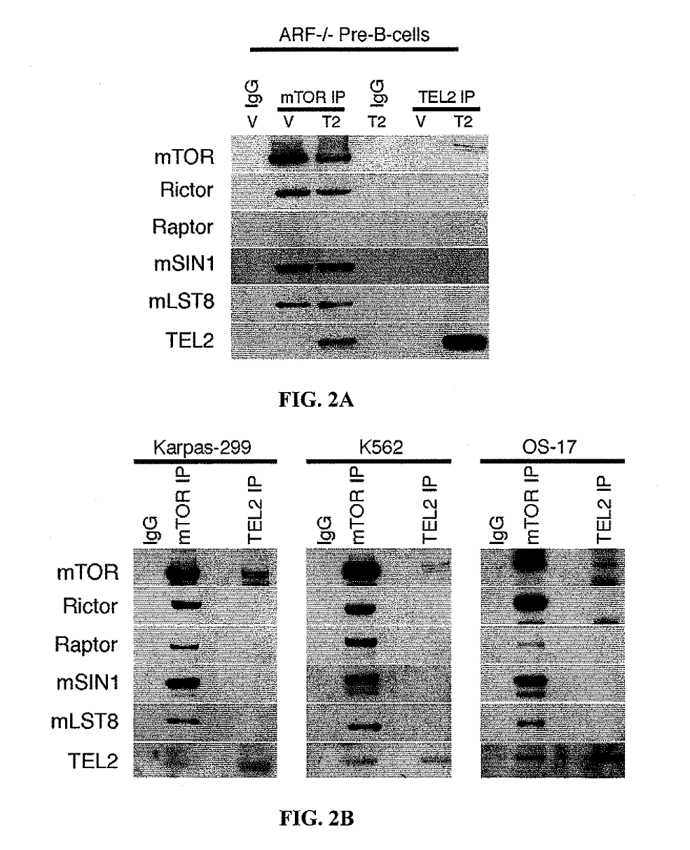

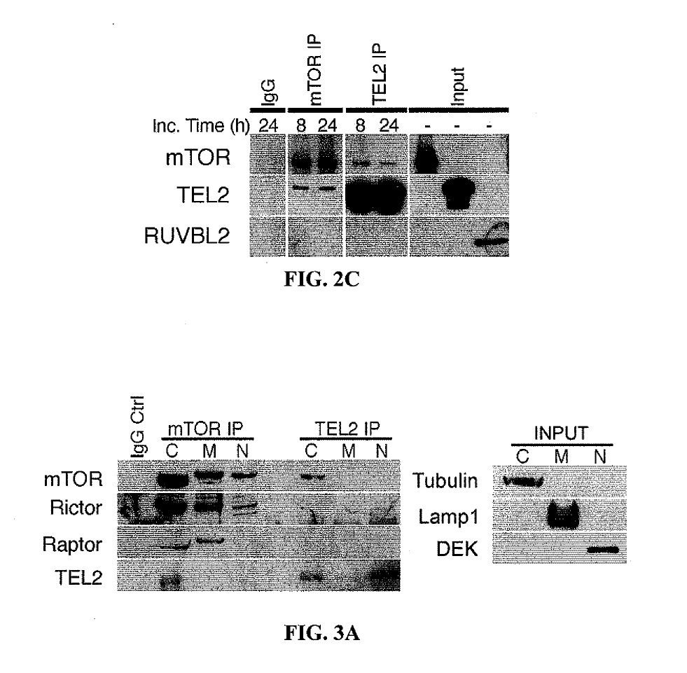

Primary mouse pre-B cells expressing TEL2/ETV7 proliferate at a much higher rate than normal pre-B cells as a result of shortened cell cycle traverse and reduced apoptosis (Cardone et al. (2005) Mol Cell Biol 25:2395). This coincides with increased phosphorylation of direct mTORC1 and both direct and indirect mTORC2 targets including p70S6K^{Thr389}, AKT^{Ser473}, NDRG1^{Thr346}, ribosomal protein S6^{Ser235/236}, 4E-BP1^{Thr37/46}, 4E-BP1^{Ser65} and 4E-BP1^{THR70} (FIG. 1). Because detection of mTORC2-activated SGK1 with the available antibodies is difficult, Phospho-NDRG1^{Thr346} was used as a bona fide readout of activated SGK1 (Garcia-Martinez and Alessi (2008) Biochem J 416:375-385). Probing the blots for total amounts of these proteins showed them to be equal. Affymetrix expression analysis verified that increased mTOR-mediated phosphorylation was not due to transcriptional upregulation of any of the known mTORC1/2 components in the TEL2 expressing pre-B cells (data not shown). Therefore, it was assessed whether increased mTOR kinase activity resulted from direct interaction of TEL2 and mTOR complexes, which was assessed with co-immunoprecipitation (co-IP) assays followed by western blot analysis using TEL2 and mTOR antibodies on whole cell lysates of mouse Arf^{-/-} pre-B cells expressing exogenous TEL2 and human cell lines expressing endogenous TEL2. mTOR antibodies co-precipitated TEL2 and TEL2 antibodies brought down mTOR in murine pre-B cells, Karpas-299 (anaplastic lymphoma), K562 (chronic myelocytic leukemia) and OS17 (osteosarcoma) cell lines (FIGS. 2A and 2B). This suggested a direct interaction between TEL2 and an mTOR complex, irrespective of whether TEL2 was expressed exogenously or endogenously. Co-immunoprecipitation experiments have been performed on eleven different, TEL2-expressing cell lines (data not shown) and invariantly mTORC3 was found, suggesting a dominant role for TEL2 in assembling the complex.

Reprobing the TEL2-immunoprecipitate/westernblots with antibodies recognizing other mTORC components showed no co-immunoprecipitation of Raptor, Rictor, SIN1, or MLST-8, whereas these proteins were present in the mTOR IPs from the same cells performed under the same experimental conditions (FIGS. 2A and 2B). These data, therefore, suggest that the mTOR-TEL2 complex (mTORC3) contains mTOR and TEL2, but none of the other mTORC1 or mTORC2 components.

To determine if co-precipitation occurred due to direct binding of TEL2 to mTOR, proteins purified from transfected HEK 293T cells were co-incubated. To ensure specificity of association, binding of TEL2 and mTOR to bacterially-expressed, purified RUVBL2, a protein not part of mTORC3, was evaluated (data not shown). IP/western blotting with mTOR and TEL2 antibodies showed that a fraction of TEL2 and mTOR co-immunoprecipitated (FIG. 2C), indicating a direct protein-protein interaction. TEL2 and mTOR antibodies did not bring down RUVBL2, confirming that the association of TEL2 and mTOR is not caused by non-specific interactions in our experimental setup.

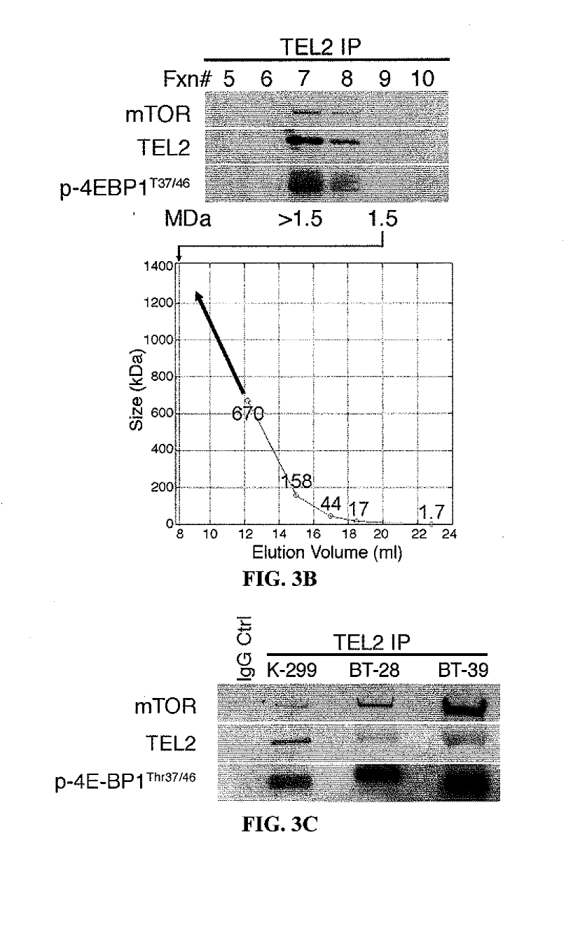

Nuclear, cytoplasmic and membrane fractions of Karpas-299 cells were subjected to IP-Western blotting to determine the cellular location of mTORC3. TEL2 was present in the nucleus and the cytoplasm, whereas mTOR was present in all three fractions (FIG. 3A); however, only cytoplasmic TEL2 co-immunoprecipitated mTOR and vice versa, demonstrating that mTORC3 is localized in the cytoplasm. In addition, Raptor and Rictor co-precipitated with mTOR in all three fractions but these proteins did not come down in the TEL2 IP, further suggesting that the protein composition of mTORC3 is distinct from that of mTORC1/2. Although TEL2 is a transcription factor that binds DNA (Potter et al. (2000) Blood 95:3341-3348), its contribution to an actively signaling mTORC3 appears to be restricted to the cytoplasm. mTOR has been localized to the nucleus in a number of cell lines (Zhang et al. (2002) J Biol Chem 277:28127-28134) and was shown to shuttle between the nucleus and cytoplasm in HEK293 cells (Kim and Chen (2000) Proc Natl Acad Sci USA 97:14340-14345). Although these results show that mTOR and TEL2 are both present in the nucleus in Karpas-299 cells, the two proteins do not co-IP in this compartment. It should also be noted that most, if not all, cytoplasmic TEL2 is associated with mTOR as shown by the near equal TEL2 signals on the western blot of the mTOR and TEL2 immunoprecipitated material from Karpas-299 cytoplasmic fractions (data not shown).

To estimate the size of mTORC3, Karpas-299 cell lysates were separated on a Superose-6 gel filtration column. The fractions were immunoprecipitated with TEL2 antibody followed by immunoblotting for mTOR, TEL2 and p-4E-BP1^{Thr37/46} (FIG. 3B). This demonstrates that TEL2 is present in two very high molecular weight fractions (>1.5 MDa), in which it co-precipitated mTOR and phosphorylated p-4E-BP1^{Thr37/46} (FIG. 3B). Therefore, mTORC3 is a large complex, likely to contain multiple other proteins. One of the proteins making up mTORC3 is phospho-4E-BP1, which is apparently recruited to mTORC3 in the absence of the Raptor scaffold protein.

To determine if mTORC3 is present in TEL2-expressing tumor xenografts, TEL2 IPs were performed, followed by mTOR immunoblotting on lysates of BT-28 (medulloblastoma) and BT-39 (glioblastoma) snap-frozen xenograft samples. This confirmed the presence of mTORC3 in the BT28 and BT-39 xenografts (FIG. 3C). To determine if TEL2 is present in the cytoplasm of TEL2-expressing human xenograft tumors (Neale et al. (2008) Clin Cancer Res 14:4572-4583), immunohistochemistry was performed on tissue sections using a TEL2 antibody. Sections of tumors were chosen with no (BT41, ependymoma), high (BT39, glioblastoma), or intermediate (BT28, medulloblastoma) levels of TEL2 RNA (data not shown). The staining intensity of the sections reflected the level of TEL2 RNA expression in the different tumors, with no staining in BT41, intermediate staining in BT28 and strong staining in BT39 (data not shown). Staining of BT39 in the presence of excess TEL2-blocking peptide produced no signal. Staining of the BT39 and BT28 xenograft tumors is predominantly cytoplasmic.

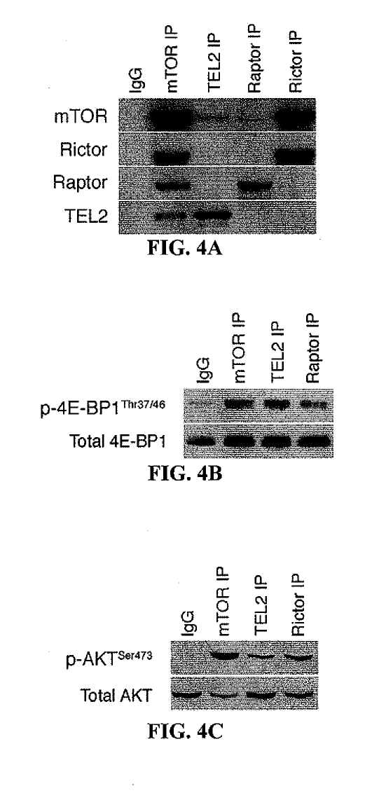

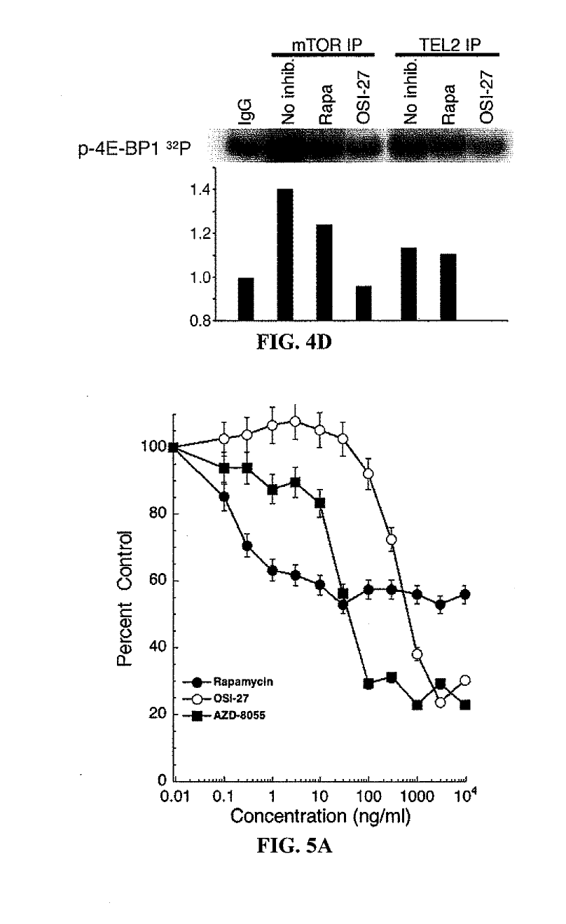

To determine the relative amounts of mTORC 1, 2 and 3, whole cell lysates of Karpas-299 cells were immunoprecipitated with non-relevant IgG, anti-mTOR, anti-TEL2, anti-Raptor, or anti-Rictor antibodies and the IPs were used for western blotting and in vitro kinase assays. Western blotting showed a two-way co-IP of TEL2 and mTOR, whereas mTOR, but not TEL2, co-precipitated with anti-Raptor or -Rictor antibodies (FIG. 4A). This blot also showed that most of the mTOR in Karpas-299 cells is present in mTORC2, much less in mTORC3, and only half that amount in mTORC1. For in vitro kinase assays, the IPs were incubated with recombinant 4E-BP1 or AKT in the presence of ATP. The phosphorylated products were immunoblotted with anti-p-4E-BP^{Thr37/46}, or p-AKT^{Ser473} antibodies demonstrating that mTORC 1 and mTORC3 phosphorylate 4E-BP1^{Thr37/46}, while mTORC2 and mTORC3 phosphorylate AKT^{Ser473} (FIGS. 4B and 4C). This showed that mTORC3 has dual mTORC 1-like and mTORC2-like activity. To address if mTORC3 kinase activity is Rapamycin sensitive, mTOR- or TEL2-immunoprecipitates from Karpas-299 cells were incubated with recombinant 4E-BP1 in the presence of γ³²P-ATP, with or without FKBP12/Rapamycin, or the mTOR ATP-competitive inhibitor OSI-27 (Garcia-Echeverria (2010) Bioorg Med Chem Lett 20:4308-4312). Adding OSI-27 to the TEL2 and mTOR IPs reduced 4E-BP1 phosphorylation to levels comparable with IPs using control IgG (FIG. 4D). However, addition of FKBP12/Rapamycin reduced the kinase activity in the mTOR IPs, but not in the TEL2 IPs. Thus, mTORC3 appears insensitive to FKBP12/Rapamycin, but sensitive to OSI-27 inhibition in this in vitro assay.

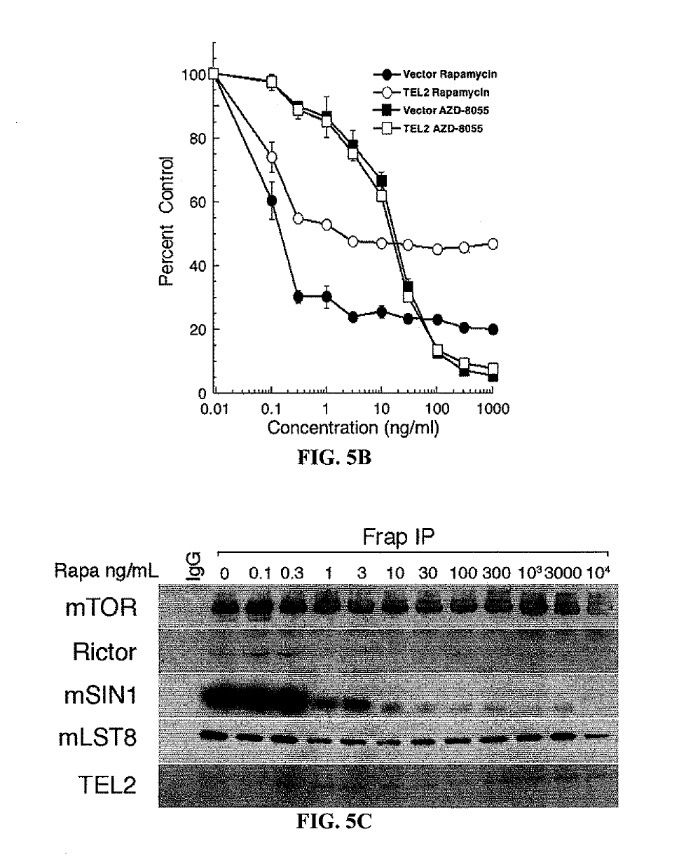

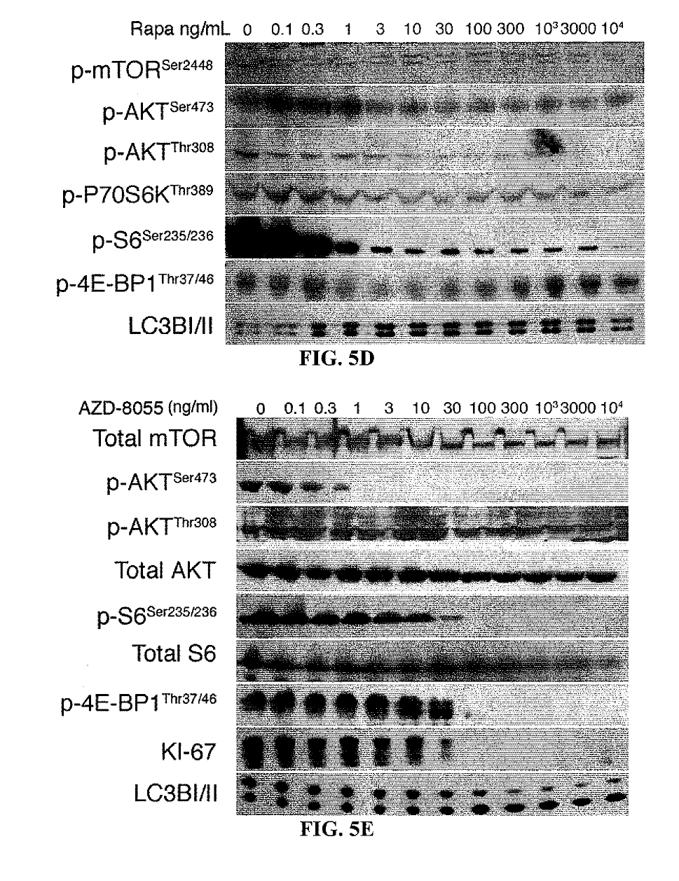

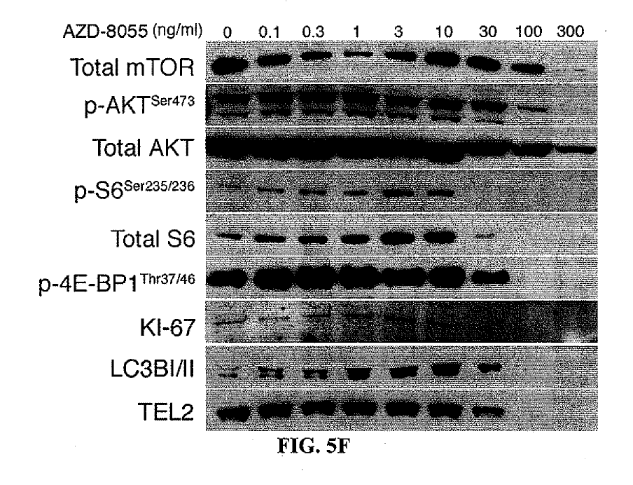

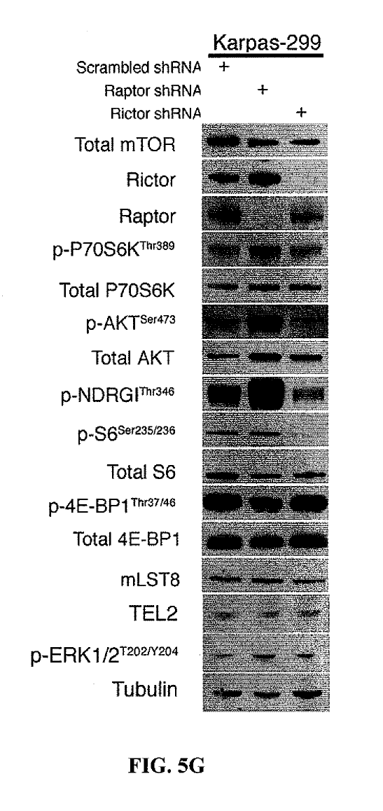

To determine if mTORC3 is also insensitive to Rapamycin inhibition in vivo, Karpas-299 cells were treated with increasing amounts of drug for three population doublings. Although this reduced their rate of proliferation (up to 1 ng/ml Rapamycin), the cells continued to proliferate at half-pace even at very high concentrations of inhibitor (up to 10⁴ ng/ml, FIG. 5A). A repeat of this experiment with mouse Art Pre-B cells demonstrated that exogenous TEL2 expression mediated resistance to Rapamycin, whereas vector-transduced Arf^{-/-} pre-B cells stopped proliferating at drug concentrations >0.3 ng/ml (FIG. 5B). Immunoblots of mTOR IPs from the Rapamycin-treated Karpas-299 cell lysates (FIG. 5C) showed dissociation of mTORC2 at concentrations >0.3 ng/ml, as assayed by the loss of co-immunoprecipitated Rictor and mSIN1. In contrast, the amount of TEL2 co-precipitating with the mTOR antibody remained unaltered, even at the highest Rapamycin concentration. Concentrations >0.3 ng/ml caused a marked reduction in the level of p-AKT^{Ser473} phosphorylation (mTORC2 inhibition), and despite the very low amount of Raptor in these cells (not shown), phosphorylation of p70S6K^{Thr389} and S6^{Ser235/236} (mTORC1 inhibition) was somewhat diminished, but then stabilized (FIG. 5D). Addition of Rapamycin did not affect the phosphorylation level of p-4E-BP1^{Thr37/46}, the protein most essential for cell proliferation and implicated in mTOR-dependent Rapamycin resistance (Armengol et al. (2007) Cancer Res 67:7551-7555; Choo et al. (2008) Proc Natl Acad Sci USA 105:17414-17419). Rapamycin also did not affect phosphorylation of mTOR^{Ser2448}, the target of p70S6K (Chiang and Abraham (2005) J Biol Chem 280:25485-25490). Another demonstration of persistent mTORC1-like signaling was the absence of autophagy, a cellular response blocked by mTORC1 signaling (Scott et al. (2004) Dev Cell 7:167-178). This was deduced from the steady relative intensities of LC3BI/II proteins at all Rapamycin concentrations (FIG. 5D).

These results show that tumor cell lines or primary cells expressing mTORC3 are resistant to Rapamycin. Karpas-299 cells treated with Rapamycin grow slower due to loss of mTORC1 and mTORC2 activity, resulting in some reduction of p-p70S6K^{Thr389} and a considerable reduction in p-S6^{Ser235/236} phosphorylation whereas p-4E-BP1^{Thr37/46} phosphorylation was maintained (FIG. 5D). In addition, Karpas-299 cells subjected to Raptor or Rictor knockdown maintained mTOR-specific p70S6K^{Thr389}, 4E-BP1^{Thr37/46}, NDRG1^{Thr364} and AKT^{Ser473} phosphorylation (FIG. 5G). This mTOR-specific signaling was not due to the reported p70S6K-IRS1-AKT feedback loop (O'Reilly et al. (2006) Cancer Res. 66:1500-8) because neither Rapamycin nor Raptor or Rictor knockdown increased ERK1/2 signaling.

In contrast to Rapamycin treatment or Raptor or Rictor knockdown, treatment of Karpas-299 cells with the selective ATP-competitive mTOR kinase inhibitors OSI-27 or AZD-8055 completely inhibited proliferation of Karpas-299 cells (FIGS. 5A and 5E). Western blot analysis of Karpas-299 lysates treated with increasing amounts of AZD-8055 showed complete loss of 4E-BP1^{Thr37/46}, P70S6K^{Thr389} and AKT^{Ser473} phosphorylation at a concentration of 100 ng/ml, which coincided with loss of cell proliferation (FIG. 5A) and transition to the G₀ phase of the cell cycle as indicated by loss of the Ki-67 antigen (FIG. 5E). Phosphorylation of AKT^{Thr308}, which is a target of membrane receptor signaling via PI3K and PDK, remained stable underlining the specificity of inhibition. At 100 ng/ml AZD-8055, the autophagy salvage pathway was activated as detected by the change in the relative amounts of the LC3BI/II proteins. Repeating the AZD-8055 experiment with TEL2-expressing primary Arf^{-/-} mouse pre-B cells mirrored the data obtained with Karpas-299 cells, as concentrations of >100 ng/ml drug completely inhibited the proliferation of the TEL2-expressing pre-B cells (FIGS. 5B and 5F), coinciding with loss of p-Akt^{Ser473}, p-S6^{Ser235/236}, p-4E-BP1^{Thr37/46} phosphorylation and loss of the KI-67 antigen. Despite the complete loss of mTOR signaling, these cells did not induce the autophagy salvage pathway and as a result, died rapidly. Together, these data showed that phosphorylation of mTOR targets is maintained by mTORC3 at Rapamycin concentrations >0.3 ng/ml, which inhibit mTORC 1/2 signaling. Also, mTORC3 signaling is fully inhibited by the selective mTOR kinase inhibitors AZD-8055 and OSI-27 at concentrations specific for mTOR kinase. The experiments with the murine pre-B cells demonstrate that TEL2 is the dominant factor setting up the mTORC3 complex, as it does so after introduction into cells that have deleted the gene. Together, these results strongly suggest that the reported Rapamycin-insensitive mTORC1 activity in human cells is not the result of a modified mTORC1 complex as has been hypothesized (Thoreen et al. (2009) J Biol Chem 284:8023-8032), but is mediated by mTORC3, which phosphorylates both mTORC 1 and mTORC2 targets.

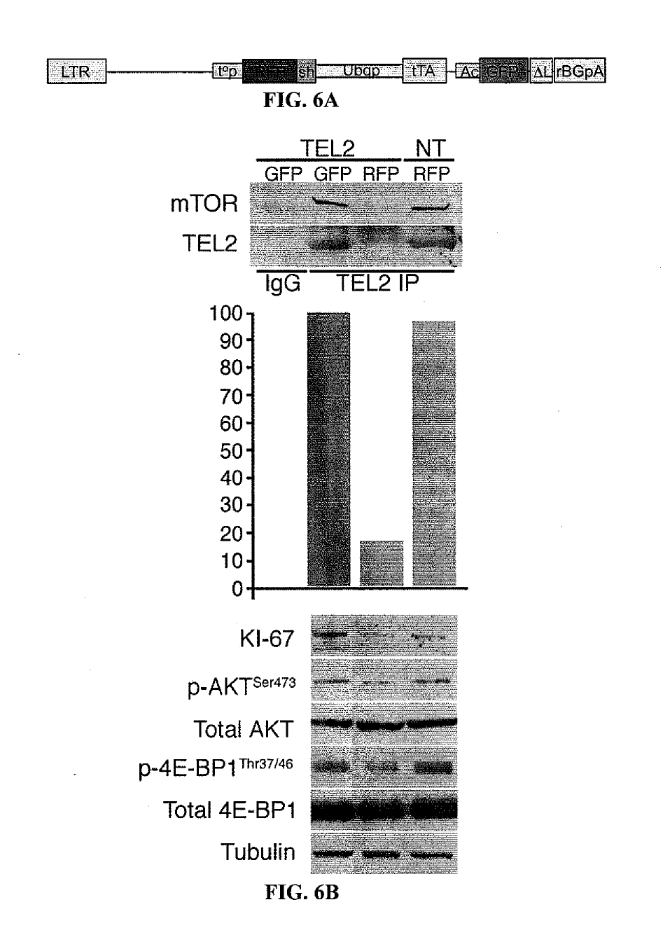

To irrefutably link endogenous TEL2 expression to cell proliferation, TEL2 knockdown experiments in OS-17 and HeLa cells were performed. OS-17 cells express robust amounts of TEL2 (FIG. 2B) while HeLa cells do not express TEL2. We transduced OS-17 and HeLa cells with a tet-on inducible TEL2 shRNA lentiviral vector (FIG. 6A) and also transduced OS-17 cells with the same lentiviral vector containing a non-targeting shRNA (NTshRNA). After sorting GFP⁺ OS-17 cells, followed by 72-hour induction with doxycycline, viable GFP⁺/RFP⁺ double-positive cells were selected by FACS. TEL2 IPs of the lysates followed by western blotting for mTOR, showed that TEL2 shRNA induction resulted in 80% knockdown of TEL2, whereas induction of NTshRNA had no effect on TEL2 expression (FIG. 6B). Similar to TEL2, mTOR co-precipitation was lost in the TEL2 knockdown cells, showing that the mTOR signal is TEL2-dependent. Immunoblots of lysates from the sorted cells showed attenuated p-pAKT^{Ser473} and p-4E-BP1^{Thr36/47} signals in the cells with knocked down TEL2 expression. Thus, TEL2 knockdown, and thereby mTORC3 knockdown, is sufficient to down-regulate proliferation and survival signaling.

Next, the effects of TEL2-shRNA or NT-shRNA induction on cell proliferation were determined. Doxycyclin-treated GFP⁺ cells were followed for 48 hours using fluorescent time-lapse microscopy (data not shown). Time-lapse images showed that bright red TEL2-shRNA-expressing OS-17 cells almost completely stopped dividing during the observation period with 45% of cells dying (data not shown) within 48 hours. Cells that failed to induce expression of TEL2 sh-RNA (green) kept dividing. Also OS-17 cells induced to express NTshRNA continued dividing (data not shown). In contrast, induction of TEL2 sh-RNA expression (bright red) in Hela cells had no effect on proliferation or survival (data not shown). The same experiment was repeated with the mTORC3-expressing DAOY medulloblastoma cell line, producing similar results (data not shown). Together, these experiments showed that knockdown of TEL2 in mTORC3-containing cell lines severely inhibited proliferation and survival, suggesting that despite continued mTORC1/2 signaling, these cell lines are addicted to mTORC3 signaling.

Expression of TEL2 has been measured in a number of human cancer cell lines including the hematopoietic cell lines Karpas-299, K562, the osteosarcoma cell lines OS-17 and the medulloblastoma cell line DAOY. This prompted an investigation of the levels of TEL2 mRNA in expression arrays of pediatric ALL (Ross et al. (2003) Blood 102:2951-2959) and AML samples (Ross et al. (2004) Blood 104:3679-3687) and a panel of pediatric solid tumor xenografts (Neale et al. (2008) Clin Cancer Res 14:4572-4583). This revealed upregulated TEL2 expression in 70% of ALL and AML samples and in 48% of solid tumor xenografts overall, including glioblastoma, medulloblastoma, neuroblastoma, rhabdomyosarcoma and rhabdoid tumors, whereas expression was low in Ewing's sarcoma and Wilm's tumor xenografts (data not shown). Additional analysis of available medulloblastoma expression arrays (Thompson et al. (2006) J Clin Oncol 24:1924-1931) showed TEL2 upregulation in 85% of cases (data not shown). Also, a recent proteomics study of human hepatocellular carcinoma identified TEL2 as one of ten upregulated proteins in this malignancy (Matos et al. (2009) J Surg Res 155:237-243). Analysis of expression array data in Oncomine (available on the world wide web at oncomine.org) showed TEL2 to be among the top 10% upregulated genes in liposarcoma, ALL, esophageal carcinoma, bladder cancer, gastric cancer, myxofibrosarcoma, breast cancer, AML-M5, colon cancer, kidney cancer, histiosarcoma, ovarian cancer, endometrial carcinoma, and medulloblastoma (Bittner (2005) International Genomics Consortium Expression Project for Oncology at Oncomine.org) (data not shown).

Publicly available cDNA array expression data of two breast cancer studies were also analyzed (van de Vijver et al. (2002) N Engl J Med 347:1999; van't Veer et al. (2002) Nature 415:530), including a total of 412 cases, using the new features of the UCSC Cancer Genomics browser (Zhu et al. (2009) Nat Methods 6:239) (data not shown). This indicated that about 30% of cases showed increased expression of TEL2, which coincided with increased expression of E2F1 and lower expression of TEF, two transcription factors for which there are binding sites in the TEL2 promoter. The tumors with higher TEL2 expression tend to be of higher grade with lymphocytic infiltration, higher frequency of BRCA1 mutation and lower frequency of ER receptor expression (data not shown). Using an affinity-purified TEL2 antibody, normal and cancerous human tissue core arrays were screened for the presence of TEL2 protein (data not shown). Most normal tissue cores (brain, thymus, tonsil, skeletal muscle, placenta, kidney, bone) did not contain detectable TEL2 protein, whereas normal pancreas showed signal in the islets of Langerhans, which could be competed with the TEL2 peptide against which the TEL2 antibody was raised. Specific signal was present in medulloblastoma cores, osteosarcoma cores, and glioblastoma cores (data not shown).

Given that TEL2-expressing cell lines and xenografts invariably contain mTORC3 and that TEL2 is frequently upregulated in different tumor types, mTORC3 must be present in many human malignancies. This conclusion is further supported by the observation that all TEL2-expressing tumor xenografts studied by Neale and coworkers (Neale et al. (2008) Clin Cancer Res 14:4572-4583) were resistant to Rapamycin treatment and that the TEL2-expressing xenografts BT28 and BT39 contained mTORC3.

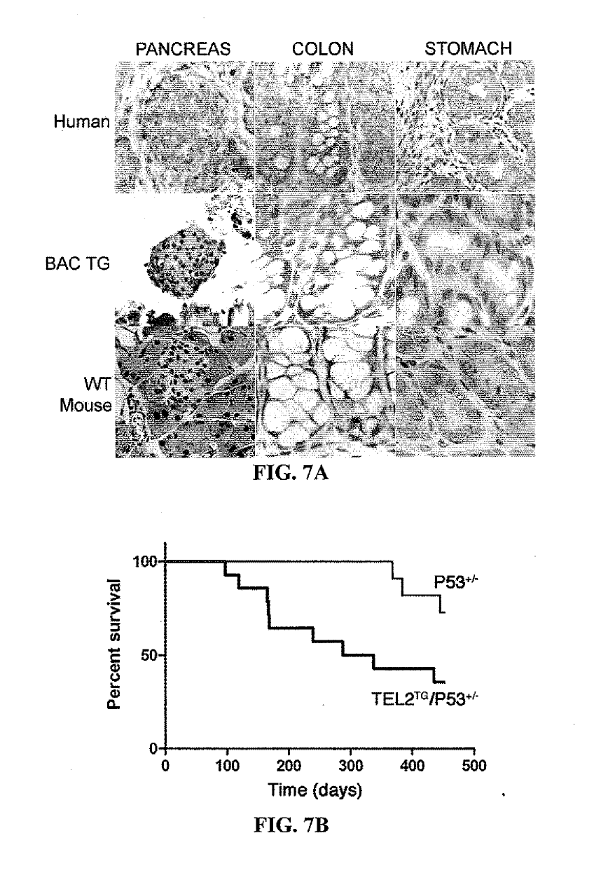

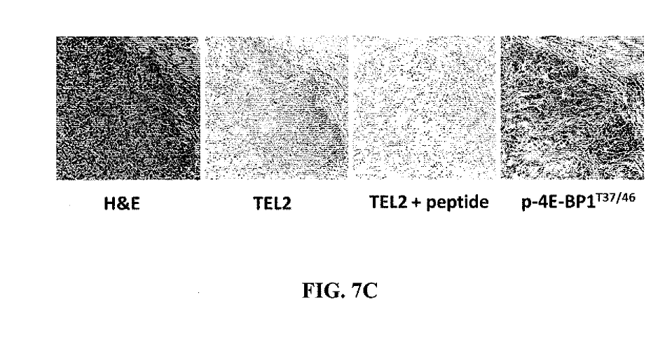

To directly link TEL2/mTORC3 expression to tumorigenesis, a transgenic (Tg) mouse carrying a single copy integration of a TEL2 BAC (TEL2 and 10 kb upstream and 30 kb downstream sequences) was generated. Immunohistochemistry of Tg mouse tissue sections confirmed that TEL2 expression in the Tg mouse tissue sections mirrored TEL2 expression in human tissue sections (FIG. 7A). TEL2-BAC^{TG+/-} mice are tumor prone late in life (>1 year) with numerous mice showing hyperplasia of the colonic crypts, another site of TEL2 expression in humans (not shown). TEL2-BAC^{TG+/-}/p53^{+/-} double mutants showed a 4-fold accelerated tumor incidence and reduced survival compared to p53^{+/-} single mutants (FIG. 7B), confirming a tumor-promoting role of the TEL2-BAC transgene. These mice have developed osteosarcoma, histiocytic sarcoma, epithelioid hemangiosarcoma, disseminated T-cell lymphoma, undifferentiated soft tissue sarcoma, and T-cell ALL. One of the osteosarcomas of a TEL2-BAC^{TG+/-}/p53^{+/-} mouse was analyzed in more detail and expressed TEL2 (FIG. 7C), with the fastest proliferating edge of the tumor showing the highest expression of TEL2. Staining of the adjacent section with p-4E-BP 1^{Thr37/46} antibody showed that the higher TEL2 expressing edge of the tumor also showed the highest levels of p-4E-BP1^{Thr37/46}, suggesting increased mTOR signaling by mTORC3. Given the high frequency of TEL2 upregulation in human tumors, it is believed that TEL2-BAC transgenic mice are a better background for modeling human tumors than wild type mice, which do not possess the Tel 2 gene.

### Materials and Methods

### Cell Lines:

To maintain maximal doubling speed and cell size, all cells were harvested at 0.3-0.4×10⁶ cells/ml or 40-60% confluency for suspension and attaching cell lines, respectively.

### Western Blotting and Co-Immunoprecipitation:

For western blot analysis, cells were lysed in 1× lysis buffer (Cell Signaling Technologies). For co-IP experiments, cells were lysed in CHAPS lysis buffer and xenografts in 1× cell signaling lysis buffer. Co-IP was performed with 2 μg antibody whilst rotating at 4° C.

### In Vitro Association:

Molar equivalents (100 ng mTOR and 13 ng TEL2) were mixed in 500 μl CHAPS lysis buffer and allowed to associate for 8 or 24 hours at 4° C. whilst rotating.

### Cell Fractionation IPs:

Sub-cellular fractions were prepared using the Qproteome Cell Compartment Kit (Qiagen) with minor alterations to the manufacturer's protocol. Each co-IP input sample was analyzed by western blotting to ensure complete separation of the relevant sub-cellular fractions.

### Gel Filtration:

Whole cell lysate of Karpas-299 cells was separated on a Superose 6 gel filtration column (GE-Healthcare). TEL2-containing fractions were immunoprecipitated with TEL2 antibody.

### IP-Kinase Assay:

Kinase assays were performed as previously described (Chiang and Abraham (2004) Methods Mol Biol 281:125-141) with minor modifications. Captured antibody-protein complexes were incubated with recombinant 4E-BP1 (Abcam) or AKT/PKB (Millipore) in the presence of (γ³²P-) ATP.

mTOR Inhibition:

Cells were plated in drug containing growth medium and followed for three population doublings.

shRNA Knockdown:

Cells were plated at 20% confluency or 200 cells/cm² in media containing 1 μg/ml Doxycyclin supplemented with 40 μm iRGD peptide (Sugahara et al. (2009) Cancer Cell 16:510-520 for western blotting and time-lapse imaging, respectively.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. An isolated mTOR complex 3 (mTORC3), wherein said mTORC3 comprises:
a) a first polypeptide comprising an mTOR polypeptide or a biologically active variant or fragment thereof; and
b) a second polypeptide comprising a TEL2 polypeptide or a biologically active variant or fragment thereof.

2. The isolated mTORC3 of claim 1, wherein said first polypeptide comprises the mTOR polypeptide of SEQ ID NO: 2, a biologically active fragment thereof, or a biologically active variant thereof having at least 80% sequence identity to the mTOR polypeptide of SEQ ID NO: 2.

3. The isolated mTORC3 of claim 1, wherein said second polypeptide comprises the TEL2 polypeptide of SEQ ID NO: 4, a biologically active fragment thereof, or a biologically active variant thereof having at least 80% sequence identity to the TEL2 polypeptide of SEQ ID NO: 4.

4. The isolated mTORC3 of claim 1, wherein said mTORC3 has a molecular weight greater than 1.5 MDa.

5. The isolated mTORC3 of claim 1, wherein said mTORC3 further comprises 4E-BP1.

6. An antibody that specifically binds to an mTOR complex 3 (mTORC3), wherein said mTORC3 comprises:
a) a first polypeptide comprising an mTOR polypeptide or a biologically active variant or fragment thereof; and
b) a second polypeptide comprising a TEL2 polypeptide or a biologically active variant or fragment thereof.

7. The antibody of claim 6, wherein said first polypeptide comprises the mTOR polypeptide of SEQ ID NO: 2, a biologically active fragment thereof, or a biologically active variant thereof having at least 80% sequence identity to the mTOR polypeptide of SEQ ID NO: 2.

8. The antibody of claim 6, wherein said second polypeptide comprises the TEL2 polypeptide of SEQ ID NO: 4, a biologically active fragment thereof, or a biologically active variant thereof having at least 80% sequence identity to the TEL2 polypeptide of SEQ ID NO: 4.

9. The antibody of claim 6, wherein said mTORC3 further comprises 4E-BP1.

10. The antibody of claim 6, wherein said antibody is a monoclonal antibody.

11. The antibody of claim 6, wherein said antibody is bispecific, wherein a first antigen binding domain specifically interacts with said first polypeptide and said second antigen binding domain specifically interacts with said second polypeptide.

12. The antibody of claim 6, wherein said antibody specifically inhibits the activity of an mTOR complex 3.

13. A mixture of a first and a second antibody comprising:
a) a first antibody having a first chemical moiety, wherein said first antibody specifically binds to a first polypeptide comprising an mTOR polypeptide or a biologically active variant or fragment thereof; and,
b) a second antibody having a second chemical moiety, wherein said second antibody specifically binds to a second polypeptide comprising a TEL2 polypeptide or a biologically active variant or fragment thereof;
wherein said first and said second chemical moiety allow for the detection of an mTOR complex 3 (mTORC3).

14. The mixture of said first and said second antibody of claim 13, wherein said first polypeptide comprises the mTOR polypeptide of SEQ ID NO: 2, a biologically active fragment thereof, or a biologically active variant thereof having at least 80% sequence identity to the mTOR polypeptide of SEQ ID NO: 2.

15. The mixture of said first and said second antibody of claim 13, wherein said second polypeptide comprises the TEL2 polypeptide of SEQ ID NO: 4, a biologically active fragment thereof, or a biologically active variant thereof having at least 80% sequence identity to the TEL2 polypeptide of SEQ ID NO: 4.

16. A compound that specifically inhibits the activity of an mTOR complex 3.

17. The compound of claim 16, wherein said compound comprises a small molecule.

18. A pharmaceutical composition comprising the antibody of claim 6 and a pharmaceutically acceptable carrier.

19. A kit for determining the level of expression of a polynucleotide encoding an mTOR polypeptide and a polynucleotide encoding a TEL2 polypeptide in a sample comprising:
a) a first polynucleotide or pair of polynucleotides capable of specifically detecting or specifically amplifying a polynucleotide encoding an mTOR polypeptide or a biologically active variant or fragment thereof; and
b) a second polynucleotide or pair of polynucleotides capable of specifically detecting or specifically amplifying a polynucleotide encoding a TEL2 polypeptide or a biologically active variant or fragment thereof;
wherein the encoded polypeptides are capable of associating with one another in an mTOR complex 3 (mTORC3).

20. The kit of claim 19, wherein
a) the first polynucleotide or pair of polynucleotides is capable of specifically detecting or amplifying a polynucleotide encoding the amino acid sequence of SEQ ID NO:2 or a sequence having at least 80% sequence identity to SEQ ID NO:2; and,
b) the second polynucleotide or pair of polynucleotides is capable of specifically detecting or amplifying a polynucleotide encoding the amino acid sequence of SEQ ID NO:4 or a sequence having at least 80% sequence identity to SEQ ID NO:4.

21. The kit of claim 19, wherein:
a) said first pair of polynucleotides comprises a first and a second primer that share sufficient sequence homology or complementarity to said polynucleotide encoding an mTOR polypeptide or biologically active variant or fragment thereof to specifically amplify said polynucleotide encoding an mTOR polypeptide or biologically active variant or fragment thereof; and
b) said second pair of polynucleotides comprises a third and a forth primer that share sufficient sequence homology or complementarity to said polynucleotide encoding an TEL2 polypeptide or biologically active variant or fragment thereof to specifically amplify said polynucleotide encoding a TEL2 polypeptide or biologically active variant or fragment thereof.

22. The kit of claim 19, wherein said kit comprises:
a) a first polynucleotide that can specifically detect said polynucleotide encoding an mTOR polypeptide or biologically active variant or fragment thereof, wherein said first polynucleotide comprises at least one DNA molecule of a sufficient length of contiguous nucleotides identical or complementary to SEQ ID NO:1; and
b) a second polynucleotide that can specifically detect said polynucleotide encoding a TEL2 polypeptide or biologically active variant or fragment thereof, wherein said second polynucleotide comprises at least one DNA molecule of a sufficient length of contiguous nucleotides identical or complementary to SEQ ID NO:3.

23. The kit of claim 19, wherein said kit comprises
a) a first polynucleotide that hybridizes under stringent conditions to the sequence of SEQ ID NO:1; and
b) a second polynucleotide that hybridizes under stringent conditions to the sequence of SEQ ID NO:3.

24. A kit for detecting the presence of an mTOR complex 3 (mTORC3) in a sample comprising an antibody of claim 6.

25. A method for detecting the level of expression of a polynucleotide encoding an mTOR polypeptide and a polynucleotide encoding a TEL2 polypeptide in a sample comprising
a) contacting said sample with
i) a first and a second primer capable of specifically amplifying a first amplicon of a polynucleotide encoding an mTOR polypeptide or a biologically active variant or fragment thereof; and,
ii) a third and a fourth primer capable of specifically amplifying a second amplicon of a polynucleotide encoding a TEL2 polypeptide or a biologically active variant or fragment thereof;
wherein the encoded polypeptides are capable of associating with one another in an mTOR complex 3 (mTORC3);
b) amplifying said first and said second amplicon; and
c) detecting said first and said second amplicon and thereby detecting the level of expression of a polynucleotide encoding an mTOR polypeptide and a polynucleotide encoding a TEL2 polypeptide in said sample.

26. The method of claim 25, wherein said first and said second primer comprise at least 8 consecutive polynucleotides of SEQ ID NO: 1 or the complement thereof, and said third and said fourth primer comprise at least 8 consecutive polynucleotides of SEQ ID NO:3 or the complement thereof.

27. A method for detecting the level of expression of a polynucleotide encoding an mTOR polypeptide and a polynucleotide encoding a TEL2 polypeptide in a sample, said method comprising:
a) contacting said sample with
i) a first polynucleotide capable of specifically detecting a polynucleotide encoding an mTOR polypeptide or a biologically active variant or fragment thereof; and,
ii) a second polynucleotide capable of specifically detecting a polynucleotide encoding a TEL2 polypeptide or a biologically active variant or fragment thereof;
wherein the encoded polypeptides are capable of associating with one another in an mTOR complex 3 (mTORC3); and
b) detecting said polynucleotide encoding the mTOR polypeptide or an active variant or fragment thereof and the polynucleotide encoding the TEL2 polypeptide or an active variant or fragment thereof.

28. A method for detecting an mTOR complex 3 (mTORC3), said method comprising:
a) contacting a sample with the antibody of claim 6; and
b) detecting a complex comprising the mTORC3 and the antibody; thereby detecting said mTORC3.

29. A method for identifying an mTOR complex 3 (mTORC3) binding agent, wherein the method comprises the steps of:
a) contacting the mTORC3 or a cell comprising the mTORC3 with a test compound; and
b) detecting a complex comprising the mTORC3 and the test compound.

30. The method of claim 29, wherein said method further comprises assaying the kinase activity of the mTORC3 to thereby determine if said test compound modulates the activity of the mTORC3 complex.

31. The method of claim 29, wherein said method further comprises contacting at least one of an mTORC1, an mTORC2, a cell comprising an mTORC1, and a cell comprising an mTORC2, and assaying for a complex comprising the mTORC 1 or mTORC2 and the test compound, thereby determining if said test compound specifically binds to the mTORC3 complex.

32. The method of claim 29, wherein said method is a cell-free method.

33. A method for screening for an mTOR complex 3 (mTORC3) antagonist, wherein said method comprises contacting mTORC3 with a test compound and assaying the kinase activity of the mTORC3 to thereby identify a compound that reduces the activity of the mTORC3.

34. The method of claim 33, wherein said method further comprises contacting at least one of an mTORC1, an mTORC2, a cell comprising an mTORC1, and a cell comprising an mTORC2, and assaying the kinase activity of the mTORC1 or mTORC2, thereby determining if said mTORC3 antagonist specifically reduces the activity of the mTORC3 complex.

35. The method of claim 29, wherein said test compound comprises an antibody.

36. The method of claim 29, wherein said test compound comprises a small molecule.

37. A method for reducing cell growth or cell survival, said method comprising contacting a cell expressing an mTOR complex 3 (mTORC3) with a specific mTORC3 antagonist.

38. The method of claim 37, wherein said specific mTORC3 complex antagonist comprises an antibody.

39. The method of claim 37, wherein said specific mTORC3 complex antagonist comprises a small molecule.

40. A method for treating or preventing a cancer in a subject in need thereof, wherein said method comprises administering to the subject a therapeutically effective amount of a specific mTORC3 complex antagonist.

41. The method of claim 40, wherein said specific mTORC3 complex antagonist comprises an antibody.

42. The method of claim 40, wherein said specific mTORC3 complex antagonist comprises a small molecule.

43. A method for diagnosing a cancer in a subject or determining the severity of a cancer in a subject, wherein said method comprises the steps of:
a) evaluating the level of an mTOR complex 3 (mTORC3) in a biological sample from said subject;
b) comparing the level of said mTORC3 in the biological sample of said subject to a control; and
c) diagnosing said cancer in said subject, wherein the level of said mTORC3 in the biological sample of said subject is relatively higher than the control; or determining the cancer of said subject is more severe than the control, wherein the level of mTORC3 in the biological sample of said subject is relatively higher than the control.

44. The method of claim 43, wherein said evaluating the level of mTORC3 in a sample of said subject comprises detecting the level of mTORC3 with an antibody of claim 6.

45. The method of claim 43, wherein said method further comprises administering to the subject a therapeutically effective amount of a specific mTORC3 complex antagonist.

46. The method of claim 40, wherein said cancer comprises a solid tumor cancer.

47. The method of claim 40, wherein said cancer comprises a pediatric cancer.

48. The method of claim 40, wherein said cancer is selected from the group consisting of acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, nephroblastoma (Wilm's tumor), hepatocellular carcinoma, esophageal carcinoma, liposarcoma, bladder cancer, gastric cancer, myxofibrosarcoma, colon cancer, kidney cancer, histiosarcoma, ovarian cancer, endometrial carcinoma, lung cancer, and breast cancer.

49. A method for treating or preventing a non-B cell cancer in a subject in need thereof, wherein said method comprises administering to the subject a therapeutically effective amount of a specific TEL2 antagonist, wherein said non-B cell cancer is selected from the group consisting of ependymoma, Ewing's sarcoma, glioblastoma, medulloblastoma, neuroblastoma, osteosarcoma, rhabdomyosarcoma, rhabdoid cancer, nephroblastoma (Wilm's tumor), esophageal carcinoma, liposarcoma, bladder cancer, gastric cancer, myxofibrosarcoma, colon cancer, kidney cancer, histiosarcoma, ovarian cancer, endometrial carcinoma, lung cancer, and breast cancer.

50. A method for diagnosing a non-B cell cancer in a subject or determining the severity of a non-B cell cancer in a subject, wherein said method comprises the steps of:
a) evaluating the expression of TEL2 in a biological sample from said subject;
b) comparing the expression of TEL2 in said biological sample of said subject with a control; and
c) diagnosing said non-B cell cancer in said subject, wherein the expression level of TEL2 in the biological sample of said subject is relatively higher than the control; or determining the non-B cell cancer of said subject is more severe than the control, wherein the expression level of TEL2 in the sample of said subject is relatively higher than the control, wherein said non-B cell cancer is selected from the group consisting of ependymoma, Ewing's sarcoma, glioblastoma, medulloblastoma, neuroblastoma, osteosarcoma, rhabdomyosarcoma, rhabdoid cancer, nephroblastoma (Wilm's tumor), esophageal carcinoma, liposarcoma, bladder cancer, gastric cancer, myxofibrosarcoma, colon cancer, kidney cancer, histiosarcoma, ovarian cancer, endometrial carcinoma, lung cancer, and breast cancer.

51. The method of claim 50, wherein said method further comprises administering to the subject a therapeutically effective amount of a specific mTORC3 complex antagonist or a specific TEL2 antagonist.

52. A method for treating an Epstein-Barr virus infection in a subject in need thereof, wherein said method comprises administering a therapeutically effective amount of a specific mTORC3 complex antagonist.

53. A non-human transgenic animal having stably incorporated into its genome a polynucleotide that encodes a TEL2 polypeptide or a biologically active variant or fragment thereof, wherein said polynucleotide is heterologous to the genome.

54. The non-human transgenic animal of claim 53, wherein said polynucleotide encodes the TEL2 polypeptide of SEQ ID NO: 4, a biologically active fragment thereof, or a biologically active variant thereof having at least 80% sequence identity to the TEL2 polypeptide of SEQ ID NO: 4.

55. The non-human transgenic animal of claim 53, wherein said non-human transgenic animal comprises a single copy of the stably incorporated polynucleotide.

56. The non-human transgenic animal of claim 53, wherein said polynucleotide encoding the TEL2 polypeptide further comprises a TEL2 promoter.

57. The non-human transgenic animal of claim 53, wherein said non-human transgenic animal is heterozygous for a p53 mutation that inhibits p53 activity.

58. The non-human transgenic animal of claim 53, wherein said non-human transgenic animal is a rodent.

59. A pharmaceutical composition comprising the mixture of a first and a second antibody of claim 13 and a pharmaceutically acceptable carrier.

60. A pharmaceutical composition comprising the compound of claim 16 and a pharmaceutically acceptable carrier.

61. The method of claim 43, wherein evaluating the level of mTORC3 in a sample of said subject comprises detecting the level of mTORC3 with the mixture of a first and a second antibody of claim 13.

62. The method of claim 43, wherein said cancer comprises a solid tumor cancer.

63. The method of claim 43, wherein said cancer comprises a pediatric cancer.

64. The method of claim 43, wherein said cancer is selected from the group consisting of acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, nephroblastoma (Wilm's tumor), hepatocellular carcinoma, esophageal carcinoma, liposarcoma, bladder cancer, gastric cancer, myxofibrosarcoma, colon cancer, kidney cancer, histiosarcoma, ovarian cancer, endometrial carcinoma, lung cancer, and breast cancer.
